(19) United States
(12) Patent Application Publication
(10) Pub. No.: US 2010/0204286 A1
(43) Pub. Date: Aug. 12, 2010

(54) METHOD FOR REDUCING GASTROINTESTINAL ADVERSE EFFECTS OF CYTOTOXIC AGENTS

(76) Inventors: Stephen R. Donahue, Lexington, MA (US); John J. Byrnes, Ashland, MA (US)

Correspondence Address: HARNESS, DICKEY, & PIERCE, P.L.C, 7700 Bonhomme, Suite 400, ST. LOUIS, MO, 63105, US

(21) Appl. No.: 12/704,286

(22) Filed: Feb. 11, 2010

Related U.S. Application Data
(60) Provisional application No. 61/151,907, filed on Feb. 12, 2009.

(51) Int. Cl. A61K 31/426; A61K 31/4172; A61K 31/415; A61P 35/00; A61P 1/00

(52) U.S. Cl. 514/365; 514/400; 514/406

(57) ABSTRACT

A method for moderating a gastrointestinal adverse effect induced by at least one cytotoxic agent, for example ionizing radiation and/or at least one chemotherapeutic agent, in a subject comprises administering to the subject a therapeutically effective amount of an ACE2 inhibitor or a compound selected from (S,S)-2-[1-carboxy-2-[3-(3,5-dichlorobenzyl)-3H-imidazol-4-yl]-ethylaminol-4-methylpentanoic acid (ORE1001), pharmaceutically acceptable salts thereof and prodrugs thereof. A method for treating a cancerous condition in a subject comprises administering to the subject (a) at least one cytotoxic anticancer agent, for example ionizing radiation and/or at least one chemotherapeutic agent; and (b) an ACE2 inhibitor or a compound selected from ORE1001, pharmaceutically acceptable salts thereof and prodrugs thereof in an amount effective to moderate a gastrointestinal adverse effect induced by the anticancer agent.

This application claims the benefit of U.S. provisional application Ser. No. 61/151,907 filed on Feb. 12, 2009, the disclosure of which is incorporated by reference herein in its entirety.

### FIELD OF THE INVENTION

The present invention relates to methods for reducing incidence or severity of gastrointestinal (GI) adverse effects induced by a cytotoxic agent such as ionizing radiation or a cytotoxic compound, for example a chemotherapeutic drug administered to treat cancer. The invention further relates to methods for treating a cancerous condition by radiation or chemotherapy with reduced incidence or severity of GI adverse effects induced thereby.

### BACKGROUND

### GI Effects of Cytotoxic Agents

The term “gastrointestinal” or its abbreviation “GI” herein refers to all or any part or parts of the alimentary canal or digestive tract from mouth to anus, including the upper GI tract, which includes the mouth, pharynx, esophagus and stomach, and the lower GI tract which includes the small intestine, comprising duodenum, jejunum and ileum, and the large intestine, comprising cecum (including the vermiform appendix which is a diverticulum of the cecum), colon (ascending colon, transverse colon, descending colon and sigmoid flexure) and rectum. The term “GI tract” is not strictly limited herein to stomach and intestines.

The entire GI tract is lined with epithelial tissue (mucous membrane or mucosa) that in a healthy subject is continually regenerated from rapidly dividing cells. Mucosal cells have a lifetime of a few days to a few weeks and are programmed to die through apoptosis as they are replaced by new cells. Continual rejuvenation of the mucosa in this way is essential to healthy digestive function. Inhibition of cell division in any part of the GI epithelium, but particularly in the lower GI tract, can therefore result in digestive disorders. Mucosal tissues also serve a protective function, thus inhibition of cell division in any part of the GI epithelium, but particularly in the mouth and rectum, can result in pain and discomfort that is usually associated with inflammation.

Among agents that can cause such adverse effects are cytotoxic agents such as ionizing radiation and cytotoxic compounds, which can be administered for example to treat cancer. Effectiveness of such agents in treatment of cancer is related to their toxicity to rapidly dividing cells; the very property that gives them their effectiveness is thus the basis for their adverse effects on the mucosal lining of the GI tract.

Of particular interest herein, but without limiting the scope of the present invention, are adverse effects of radiation and cytotoxic compounds in the lower GI tract (such effects herein are referred to as enteropathies and include enteritis and, where the effects are more localized in the colon or rectum, colitis or proctitis respectively) and in the oropharyngeal cavity (where the effects include oral mucositis).

Inflammatory enteropathies are well known and include inflammatory bowel disease (IBD), a class of idiopathic diseases of the digestive tract, including ulcerative colitis and Crohn's disease, that are believed to involve an autoimmune reaction. Methods for treating IBD are disclosed, for example, in U.S. Patent Application Publication Nos. 2008/0107650 and 2008/0110793, both of Tartaglia et al. Each of these publications is incorporated herein by reference in its entirety without admission that either one constitutes prior art to the present invention.

Distinct from such idiopathic diseases are enteropathic conditions induced by cytotoxic agents or influences. One such condition is radiation enteritis (also known by other names including radiation enteropathy or, where the symptoms are more limited to the colon or rectum, radiation colitis or radiation proctitis respectively), wherein damage to the mucosal lining of the intestine is induced by any form of cytotoxic radiation. Such radiation can include X-rays (although typically an X-ray dose is insufficient to provoke a cytotoxic response), as well as alpha-, beta- and gamma-rays emitted from a radioactive source. Radiation enteritis is most commonly an adverse side-effect of radiation therapy for cancer, but can also result from accidental, occupational or malign (for example through criminal or hostile activity) exposure to radiation. Radiation enteritis can be acute or chronic.

The acute form typically has an onset within days or even hours of exposure and generally persists no more than about 2-3 weeks after exposure ends (for example after the end of a course of radiation therapy). Acute radiation enteritis is characterized by nausea, vomiting and diarrhea. Particularly in the case of acute radiation-induced proctitis, inflammation of the rectal mucosa can be evident. Acute radiation enteritis is almost universal in patients receiving radiation therapy to the abdominal or pelvic region, for example in treatment of genitourinary cancers such as cancers of the prostate, cervix or bladder.

Chronic radiation enteritis is more insidious and often does not become evident for several months, or even a year or more, following exposure. It generally involves inflammation of the intestinal mucosa that, often with sequelae such as collagen deposition and/or fibrosis, can seriously compromise intestinal function, leading to symptoms such as wave-like abdominal pain, bloody diarrhea, frequent urges to defecate, fatty stools, weight loss, nausea and vomiting. In severe cases narrowing (stricture or stenosis) of the intestinal canal, sometimes resulting in complete obstruction, can occur. Perforation of the intestinal wall and rectal bleeding can also occur. Chronic radiation enteritis often requires surgery and is occasionally fatal. It is estimated that about 5-15% of patients receiving abdominal or pelvic radiation therapy develop chronic radiation enteritis. Chronic enteritis is believed to be, at least in part, a complication arising from acute enteritis, although the link is not well understood. More detailed information can be found, for example, in review articles such as those individually cited below and incorporated herein by reference.

Johnson & Carrington (1992) Clin. Radiol. 45:4-12.

MacNaughton (2000) Aliment. Pharmacol. Ther. 14:523-528.

Cytotoxic agents other than radiation that can induce enteritis include cytotoxic drugs such as alkylating and platinum agents administered, for example, as chemotherapeutics for treatment of cancer. Whereas radiation therapy typically leads to enteritis only if the focus of the therapy is in the abdominal or pelvic region, chemotherapeutic drugs can be distributed systemically in the body and cause enteritis regardless of the locus of the cancer being treated.

Inflammation in radiation- or chemotherapy-induced enteritis can be reduced, at least temporarily, with anti-inflammatory agents such as corticosteroids. Corticosteroids may be administered by a variety of routes depending on the location and severity of disease; for example they may be administered intravenously (e.g., methylprednisolone, hydrocortisone), orally (e.g., prednisone, prednisolone, budesonide, dexamethasone), or topically (enema, suppository or foam preparations, particularly in the case of proctitis).

Corticosteroids do not address the underlying cause of enteritis; furthermore the potential complications of corticosteroid use are multiple and include fluid and electrolyte abnormalities, osteoporosis, aseptic necrosis, peptic ulcers, cataracts, neurologic and endocrine dysfunctions, infectious complications, and occasional psychiatric disorders (including psychosis). Infectious complications can be particularly severe when corticosteroids are used to treat cytotoxic injury to the GI tract since the epithelial layer that is damaged by exposure to cytotoxic agents is the barrier that protects the body from a multitude of bacteria and other infectious agents.

Oral mucositis is a debilitating inflammatory disease of the oral mucosa, often manifested as erythema and painful ulcerative lesions of the mouth, in some cases also affecting the throat (oropharyngeal mucositis). Oral mucositis is a well-known complication of cancer therapies involving radiation therapy and/or chemotherapy, occurring in about 40% of patients receiving such therapies. See, for example, Best Practice 2(3) (1998) (www.oralcancerfoundation.org/dental/pdf/mucositis.pdf).

Oral mucositis is defined by the National Cancer Institute (NCI) as inflammation of oral mucosa resulting from chemotherapeutic agents or ionizing radiation, and as a type of stomatitis, which refers generally to inflammation of oral tissue, including mucosa, dental periapices and periodontium. See, for example, www.cancer.gov/cancertopics/pdq/supportivecare/oralcomplications/HealthProfessional/page5.

Oral mucositis can result from systemic effects of cytotoxic chemotherapy agents and from local effects of radiation therapy. It has become a common and often treatment-limiting side effect of therapy for cancers, particularly but not exclusively for cancers of the head and neck, especially where the therapy includes radiation. Typically, oral mucositis develops within 7 to 14 days after initiation of chemotherapy or radiation therapy. Commonly inflammation of the oral mucosa leads to acute xerostomia (dry mouth). Xerostomia can also occur chronically as a result of fibrosis of the salivary gland. Long-term consequences of oral mucositis and xerostomia can include debilitating discomfort and pain, reduced ability to eat and speak, and increased susceptibility to secondary diseases such as oral infections, dental caries and periodontal disease.

Few interventions have proven effective for treatment (including prophylaxis) of oral mucositis. Palliative care is standard in management of oral mucositis, and can include
bland rinses, for example with 0.9% saline solution, sodium bicarbonate solution or 0.9% saline/sodium bicarbonate solution;
topical anesthetics, for example viscous formulations, ointments and sprays comprising lidocaine; sprays or gels comprising benzocaine; 0.5% or 1% dyclonine hydrochloride; or diphenhydramine solution;
mucosal coating agents, for example aluminum hydroxide suspension; bismuth subsalicylate suspension; products containing film-forming agents; or bioadherent oral gels;
analgesics, for example benzydamine hydrochloride topical rinse or opioid drugs administered orally, intravenously (e.g., bolus, continuous infusion, patient-controlled analgesia), transdermally by patch, or transmucosally;
growth factor, for example keratinocyte growth factor 1 (e.g., palifermin, specifically to decrease incidence and duration of severe oral mucositis in patients undergoing high-dose chemotherapy with or without radiation therapy followed by bone marrow transplant for hematological cancers).

Also helpful are good oral hygiene practices and cryotherapy (for example by means of ice chips placed in the mouth). Various mouthwashes have also been used. Most institutions have their own version of a “magic mouthwash” which is typically a preparation containing lidocaine, diphenhydramine and a coating agent such as aluminum hydroxide.

International Patent Publication No. WO 02/41837 mentions methods for treating mucositis comprising contacting a mucosal site, for example an oral mucosal site, with a composition comprising a pharmaceutical substance and a biocompatible polymer (e.g., poloxamer 407) that adheres to the mucosal site. Among pharmaceutical substances mentioned as being useful are antioxidants, antibacterials, anti-inflammatories, anesthetics, analgesics, proteins, peptides and cytokines.

Oral mucositis, particularly when severe, has a major impact on daily functioning, well-being and quality of life of a patient. It can also compromise a patient's ability to tolerate planned cancer therapy, resulting in missed doses or dose reductions, and can thereby lead to a less successful outcome of such therapy, for example greater likelihood of recurrence of the cancer, shorter remission, or increased mortality.

Effective, tolerable therapies for radiation-induced and chemotherapy-induced GI disorders including enteritis and oral mucositis are a long-felt need in the art. A recent development that has shown promise is use of amifostine, a thiol prodrug that upon dephosphorylation provides a metabolite that protects normal tissues from cytotoxic agents, particularly ionizing radiation. Dephosphorylation occurs to a relatively limited extent in tumors, thus amifostine does not strongly inhibit the anticancer efficacy of radiotherapy. See, for example, the review article by Kouvaris et al. (2007) The Oncologist 12:738-747.

There remains a need for additional radioprotective and chemoprotective treatments to moderate or mitigate adverse effects of anticancer therapies in the GI tract.

### BACKGROUND OF THE INVENTION

Inflammatory activity in the GI tract is known to involve activation of nuclear factor κB (NF-κB). See, e.g., Schreiber et al. (1998) Gut 42:477-484, concluding that in IBD, particularly in Crohn's disease, increased activation of NF-κB may be involved in regulation of the inflammatory response, and that inhibition of NF-κB activation may represent a mechanism by which steroids exert an anti-inflammatory effect.

Further, the anti-TNFα antibody infliximab has been reported to decrease NF-κB activity in Crohn' s disease (see Guidi et al. (2005) Int. J. Immunopathol. Pharmacol. 18(1):155-164).

Conversely, an increase in NF-κB activity has been reported to precede relapse of symptoms in Crohn's disease patients exhibiting failure to maintain response to infliximab (see Nikolaus et al. (2000) Lancet 356(9240):1475-1479).

The NF-κB signaling pathway is involved in a wide range of pro-inflammatory effects. See, e.g., Schreiber et al. (1998), supra. Angiotensin II (Ang II), a member of the renin-angiotensin system (RAS) and the primary product of angiotensin converting enzyme (ACE), is known to exert pro-inflammatory effects in a variety of tissues, via its type 1 and type 2 receptors (AT₁ and AT₂ respectively) and, in many cases, ultimately through activation of NF-κB, as indicated below.

In the classical pathway of Ang II synthesis in the circulating RAS, the precursor of Ang II is angiotensinogen, which is principally produced in the liver and then cleaved by renin to form angiotensin I (Ang I), which is converted by ACE into Ang II that is carried to various target cells via the circulatory system. See, e.g., Inokuchi et al. (2005) Gut 54:349-356, and sources cited therein. In addition, tissue-specific renin-angiotensin systems have been identified in many organs, suggesting that various tissues have the ability to synthesize Ang II independently of circulating RAS, including kidney, brain, aorta, adrenal gland, heart, stomach and colon.

Donoghue et al. (2000) Circ. Res. 87:1-9 reported identification of a carboxypeptidase related to ACE from sequencing of a human heart failure ventricle cDNA library. This carboxypeptidase, ACE2, was stated to be the first known human homolog of ACE. The authors further reported that the metalloprotease catalytic domains of ACE2 and ACE are 42% identical, and that, in contrast to the more ubiquitous ACE, ACE2 transcripts are found only in heart, kidney, and testis in the 23 human tissues examined.

U.S. Pat. No. 6,194,556 to Acton et al. discloses novel genes encoding ACE2. Therapeutics, diagnostics and screening assays based on these genes are also disclosed.

Harmer et al. (2002) FEBS Lett. 532:107-110 reported quantitative mapping of the transcriptional expression profile of ACE2 (and the two isoforms of ACE) in 72 human tissues. The study reportedly confirmed that ACE2 expression is high in renal and cardiovascular tissues. It was further reported that ACE2 shows comparably high levels of expression in the gastrointestinal system, in particular in ileum, duodenum, jejunum, cecum and colon. The authors proposed that in probing functional significance of ACE2, some consideration should be given to a role in gastrointestinal physiology and pathophysiology.

Rice et al. (2003) Bull. Br. Soc. Cardiovasc. Res. 16(2):5-11 reviewed potential functional roles of ACE2 and indicated that its expression is mainly localized in testis, kidney, heart and intestines.

Ferreira & Santos (2005) Braz. J. Med. Biol. Res. 38:499-507 have summarized important pathways of the RAS, including roles of ACE and ACE2, as shown in FIG. 1 herein.

As evidence of implication of Ang II, the main product of ACE, in a variety of pro-inflammatory effects, see for example:
Phillips & Kagiyama (2002) Curr. Opin. Investig. Drugs 3(4):569-577, who reviewed literature showing Ang II to be a key factor, via NF-κB activation, in promoting inflammation, inter alia, in atherosclerosis;
Costanzo et al. (2003) J. Cell Physiol. 195(3):402-410, who reported up-regulation by Ang II of endothelial cell adhesion molecules involved in atherosclerosis, via inflammatory cytokines through NF-κB activation;
Sanz-Rosa et al. (2005) Am. J. Physiol. Heart Circ. Physiol. 288:H111-H115, who reported that blocking the AT₁ receptor reduces the level of vascular and circulating inflammatory mediators such as NF-κB and TNF-α in spontaneous hypertension;
Esteban et al. (2004) J. Am. Soc. Nephrol. 15:1514-1529, who reported that Ang II, via AT₁ and AT₂, activates NF-κB and thereby promotes inflammation in obstructed kidney; and
Inokuchi et al. (2005), supra, who reported that in angiotensinogen gene knockout mice, which have low levels of Ang II, inflammatory colitis induced by 2,4,6-trinitrobenzenesulfonic acid (TNBS) is ameliorated, and that blocking the AT₁ receptor also ameliorated TNBS-induced colitis.

Antagonism of the RAS has been postulated as a prophylactic strategy for immune-mediated inflammatory bowel disease (Inokuchi et al. (2005), supra).

The proinflammatory effects of the ACE product Ang II have been found to be generally counterbalanced by ACE2 in various studies involving ACE2 disruption and/or mutants lacking the ACE2 gene. See for example:
Crackower et al. (2002) Nature 417(6891):822-828, who reported that disruption of ACE2 or deletion of the ACE2 gene in various rat models raises the level of Ang II;
Huentelman et al. (2005) Exp. Physiol. 90(5):783-790, who reported that injection of a vector encoding ACE2 protects wild-type mice against Ang II-induced cardiac hypertrophy and fibrosis; and
Imai et al. (2005) Nature 436(7047):112-116, who reported that deletion of the ACE gene or giving ACE2 protein to wild-type mice protects against acid-induced acute lung injury.

The primary product of ACE2, namely angiotensin (1-7), via its receptor (Mas), has generally been found to oppose functions of the ACE product Ang II. See for example:
Guy et al. (2005) Biochim. Biophys. Acta 1751(1):2-8, who reviewed literature indicating inter alia that ACE2 regulates heart and kidney function by control of Ang II levels relative to angiotensin (1-7), and may therefore counterbalance the effects of ACE within the RAS;
Ferreira & Santos (2005), supra, who reviewed literature indicating inter alia that

ACE inhibitor benefits may be partly mediated by the ACE2 product angiotensin (1-7), plasma levels of which are greatly increased following chronic administration of ACE inhibitors;
Mendes et al. (2005) Regul. Pept. 125(1-3):29-34, who reported that infusion of angiotensin (1-7) reduces Ang II levels in the heart and postulated that such reduction may contribute to beneficial effects of angiotensin (1-7); and
Tallant & Clark (2003) Hypertension 42:574-579, who reported that angiotensin (1-7) reduces smooth muscle growth after vascular injury, and counteracts stimulation by Ang II of growth and mitogen activated protein (MAP) kinase activity in rat aortic vascular smooth muscle cells.

Thus low levels of Ang II appear to ameliorate inflammatory colitis (Inokuchi et al. (2005), supra), and ACE2 activity appears to counterbalance inflammatory effects of Ang II in a variety of tissues, whether by increasing angiotensin (1-7) levels or reducing Ang II levels or both.

In one scenario, therefore, promotion of ACE2 activity might be of interest for reducing inflammation in diseases such as enteritis and mucositis. Huentelman et al. (2004) Hypertension 44:903-906 proposed, similarly, that in vivo activation of ACE2 could lead to protection and successful treatment for hypertension and other cardiovascular diseases, by counterbalancing the potent vasoconstrictive effects of Ang II.

Above-cited U.S. Pat. No. 6,194,556, which discloses novel genes encoding ACE2, proposes at column 60, lines 36-54 that: “Yet other diseases or conditions in which bradykinin is overproduced and in which ACE-2 agonist therapeutics capable of inactivating bradykinin can be useful include pathological conditions such as septic and hemorrhagic shock, anaphylaxis, arthritis, rhinitis, asthma, inflammatory bowel disease, sarcoidosis, and certain other conditions including acute pancreatitis, post-gastrectomy dumping syndrome, carcinoid syndrome, migraine, and hereditary angioedema” (references omitted).

Agents that inhibit rather than promote ACE2 activity have been described in the art. For example, Huentelman et al. (2004), supra, reported efforts to identify ACE2 inhibitory compounds that inhibit infection by SARS-CoV, the coronavirus responsible for severe acute respiratory syndrome (SARS), for which ACE2 has been found to be a functional receptor. Among the compounds so identified was NAAE (N-(2-aminoethyl)-1-aziridine-ethanamine).

U.S. Pat. No. 6,900,033 to Parry et al. discloses peptides comprising specific amino acid sequences that are said to bind specifically to ACE2 protein or ACE2-like polypeptides. It is proposed at column 53, lines 63-65 thereof that “an abnormally high a[n]giotensin II level could result from abnormally low activity of ACE-2” and at column 63, lines 21-32 thereof that “ACE-2 binding polypeptides . . . which activate ACE-2-induced signal transduction can be administered to an animal to treat, prevent or ameliorate a disease or disorder associated with aberrant ACE-2 expression, lack of ACE-2 function, aberrant ACE-2 substrate expression, or lack of ACE-2 substrate function. These ACE-2 binding polypeptides may potentiate or activate either all or a subset of the biological activities of ACE-2-mediated substrate action . . . ”. Further, at column 71, lines 26-37 thereof, polypeptides “of the invention” (whether activating or inhibitory not specified) inter alia are said to be useful “to treat, prevent, or ameliorate inflammation, including, but not limited to, inflammation associated with infection (e.g., septic shock, sepsis, or systemic inflammatory response syndrome (SIRS)), ischemia-reperfusion injury, polytrauma, pain, endotoxin lethality, arthritis (e.g., osteoarthritis and rheumatoid arthritis), complement-mediated hyperacute rejection, nephritis, cytokine- or chemokine-induced lung injury, inflammatory bowel disease, Crohn's disease, and resulting from over production of cytokines (e.g., TNF or IL-1).” Separately, ACE2 binding peptides that are reported to inhibit ACE2 in vitro are identified in Table 2 at columns 127-130 thereof.

Huang et al. (2003) J. Biol. Chem. 278(18):15532-15540 reported that one such ACE2 inhibitory peptide, namely DX600, exhibited an ACE2 K, value of 2.8 nM.

Li et al. (2005) Am. J. Physiol. Renal Physiol. 288:F353-F362 reported that DX600 blocked Ang I-mediated generation of angiotensin (1-7) in rat nephron segments.

U.S. Pat. No. 6,632,830 to Acton et al. discloses compounds comprising a zinc coordinating moiety and an amino acid mimicking moiety, said to be useful for modulating activity of ACE2. More particularly, there are disclosed ACE2 inhibiting compounds of a generic formula presented therein. Such compounds are said to be useful for treating an “ACE-2 associated state” in a patient. “ACE-2 associated states” are said to include high blood pressure and diseases and disorders related thereto, in particular arterial hypertension, congestive heart failure, chronic heart failure, left ventricular hypertrophy, acute heart failure, myocardial infarction and cardiomyopathy; states associated with regulating smooth cell proliferation, in particular smooth muscle cell proliferation; kidney diseases and disorders; other hyperadrenergic states; kinetensin associated conditions including those caused by, or contributed to by, abnormal histamine release, for example in local or systemic allergic reactions including eczema, asthma and anaphylactic shock; infertility or other disorders relating to gamete maturation; cognitive disorders; disorders associated with bradykinin and des-Arg bradykinin; and “other examples” (column 36, lines 58-67 thereof) that are said to include “SIRS . . . , sepsis, polytrauma, inflammatory bowel disease, acute and chronic pain, bone destruction in rheumatoid and osteo arthritis and periodontal disease, dysmenorrhea, premature labor, brain edema following focal injury, diffuse axonal injury, stroke, reperfusion injury and cerebral vasospasm after subarachnoid hemorrhage, allergic disorders including asthma, adult respiratory distress syndrome, wound healing and scar formation.”

Dales et al. (2002) J. Am. Chem. Soc. 124:11852-11853 reported ACE2 IC₅₀ values of a range of such compounds. The most active of these was compound 16, identified therein as having the formula

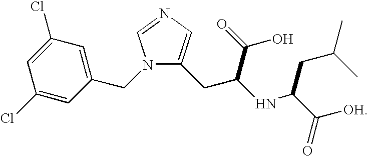

All four stereoisomers of compound 16 were prepared, and the greatest potency was reported for the S,S-isomer, which reportedly had an IC₅₀ for ACE2 of 0.44 nM. The S,S-isomer of the above compound, 2-[1-carboxy-2-[3-(3,5-dichlorobenzyl)-3H-imidazol-4-yl]-ethylamino]-4-methylpentanoic acid, is referred to herein as ORE1001 and has previously been referred to as GL1001 or MLN-4760.

U.S. Patent Application Publication No. 2004/0082496 of Acton et al. discloses additional compounds said to be useful for modulating activity of ACE2. Methods of using the inhibitors and pharmaceutical compositions containing the inhibitors to treat a body weight disorder, to decrease appetite, to increase muscle mass, to decrease body fat, to treat diabetes and to treat a state associated with altered lipid metabolism, are also described.

### SUMMARY OF THE INVENTION

The present invention derives in part from an experimental finding that could not reasonably have been predicted. This finding, described in detail in Example 5 hereof, is that in an art-accepted rat model of radiation-induced proctitis, ORE1001 reduces severity of proctitis and of various histopathological indicators of proctitis, especially when administration of the ORE1001 begins before exposure to radiation.

In accordance with this finding, there is now provided a method for moderating a GI adverse effect induced by exposure to at least one cytotoxic agent in a subject, comprising administering to the subject a therapeutically effective amount of a compound selected from the group consisting of ORE1001, pharmaceutically acceptable salts thereof and prodrugs thereof.

There is further provided a method for treating a cancerous condition in a subject, comprising administering to the subject (a) at least one cytotoxic anticancer agent and (b) a compound selected from the group consisting of ORE1001, pharmaceutically acceptable salts thereof and prodrugs thereof in an amount effective to moderate a GI adverse effect induced by the anticancer agent.

There is still further provided a therapeutic combination comprising (a) at least one cytotoxic anticancer agent and (b) a compound selected from the group consisting of ORE1001, pharmaceutically acceptable salts thereof and prodrugs thereof in an amount effective, when administered to a subject receiving the anticancer agent, to moderate a GI adverse effect induced in the subject by the anticancer agent.

In various embodiments, the at least one cytotoxic agent comprises ionizing radiation, at least one cytotoxic chemical agent (e.g., a chemotherapeutic agent) or a combination thereof.

In various embodiments, the GI adverse effect moderated comprises enteritis and/or oral mucositis.

In various embodiments, administration of the compound begins before, simultaneously with or after first exposure to the cytotoxic agent, for example in a course of radiation therapy or chemotherapy.

It is believed, without being bound by theory, that the GI adverse effect moderating properties of ORE1001 are mediated at least in part by inhibition of ACE2, and that any ACE2 inhibitor can be useful according to the present invention.

Accordingly, there is still further provided a method for moderating a GI adverse effect induced by exposure to at least one cytotoxic agent in a subject, comprising administering to the subject a therapeutically effective amount of an ACE2 inhibitor.

There is still further provided a method for treating a cancerous condition in a subject, comprising administering to the subject (a) at least one cytotoxic anticancer agent and (b) an ACE2 inhibitor in an amount effective to moderate a GI adverse effect induced by the anticancer agent.

There is still further provided a therapeutic combination comprising (a) at least one cytotoxic anticancer agent and (b) an ACE2 inhibitor in an amount effective, when administered to a subject receiving the anticancer agent, to moderate a GI adverse effect induced in the subject by the anticancer agent.

Other embodiments, including particular aspects of the embodiments summarized above, will be evident from the detailed description that follows.

### BRIEF DESCRIPTION OF THE DRAWINGS

FIG. 1 is a schematic representation of enzymatic pathways of the renin-angiotensin system (RAS) involved in generation of angiotensin peptides. Key:

ACE=angiotensin converting enzyme;

AMP=aminopeptidase;

Ang=angiotensin;

AT₁=angiotensin II type 1 receptor;

AT₁₋₇=angiotensin (1-7) receptor;

AT₂=angiotensin II type 2 receptor;

D-Amp=dipeptidyl aminopeptidase;

TRAP=insulin regulated aminopeptidase;

NEP=neutral endopeptidase 24.11;

PCP=prolyl carboxypeptidase;

PEP=prolyl endopeptidase.

(From Ferreira & Santos (2005), supra.)

FIG. 2 is a graphical representation of inhibition by ORE1001 of TNFα-induced activation of NF-κB in recombinant HeLa reporter cells, as described in Example 2.

FIG. 3 is a graphical representation of inhibition by ORE1001 of in vivo basal NF-κB-dependent transcription in recombinant reporter mice, as described in Example 3.

FIG. 4 is a graphical representation of inhibition by ORE1001 of in vivo LPS-induced NF-κB signaling in mice, as described in Example 4. Mice were pretreated with ORE1001 (subcutaneous) for 1 hour before LPS treatment. All mice treated with 0.1 mg/kg LPS (i.v.). Abdominal ROI used for quantitative data (2.76×3.7 cm). Mean±SEM, n=5 for each group; *p<0.05, **p<0.01, ANOVA and student t-test between treatments and controls.

FIG. 5 is a graphical representation of inhibition by ORE1001 of in vivo LPS-induced NF-κB signaling in mice, as described in Example 4. Male mice were pretreated with ORE1001 and LPS, and were imaged, as in FIG. 4. Mean±SEM, n=5 for each group; *p<0.05, **p<0.01, ANOVA and student t-test between treatments and controls.

FIG. 6 is a graphical representation of inhibition by ORE1001 of LPS-induced NF-κB-dependent transcription in selected organs of recombinant reporter mice, as described in Example 4.

FIG. 7 is a graphical representation of reduction in severity of radiation-induced proctitis in rats by administration of ORE1001, as described in Example 5. Probability (p) values are versus vehicle control.

FIG. 8 is a graphical representation of effects of ORE1001 on histopathology of rats having radiation-induced proctitis, as described in Example 5. *p<0.05 versus vehicle control.

### DETAILED DESCRIPTION

In a first embodiment of this invention, a method is provided for moderating a GI adverse effect induced by exposure to at least one cytotoxic agent in a subject.

The at least one cytotoxic agent can comprise, for example, ionizing radiation, at least one cytotoxic chemical agent or a combination thereof.

The term “moderating” in the present context means ameliorating, mitigating or reducing incidence or severity of the adverse effect and can include, in ideal situations, complete suppression or correction of the adverse effect. The adverse effects, as noted herein, can be extremely debilitating or even life-threatening, thus even a modest degree of moderation of such effects can bring great benefit to the subject. The adverse effect to be moderated can be one already being experienced by the subject, for example due to past or ongoing exposure to a cytotoxic agent. Alternatively, the adverse effect to be moderated can be one to which the subject is at identifiable risk, i.e., an effect that is anticipated with a reasonable degree of probability, for example in a subject scheduled to undergo anticancer therapy with radiation and/or a cytotoxic chemotherapeutic agent.

An adverse effect “induced” by exposure to a cytotoxic agent is one that arises at least in part as a direct or indirect result or consequence of such exposure. Induction herein includes exacerbation of a pre-existing condition, for example an inflammatory condition of the GI tract, as well as de novo initiation of an adverse condition, as a direct or indirect result of such exposure.

Adverse effects of particular interest herein are those attendant upon inhibition of natural mucosal regeneration in the GI tract. Cytotoxic agents generally inhibit or interfere with normal processes of cell division, for example through DNA damage, and are therefore especially injurious to tissues that depend on continuous regeneration, such as mucosal tissues. Adverse effects of cytotoxic agents in the GI tract are manifested histologically by inflammation, edema and necrosis, followed by scarring or fibrosis that can lead to partial or total obstruction of the GI tract. As noted above, such effects can be acute or chronic.

The term “exposure” herein is to be interpreted broadly. Exposure can be accidental or intentional. Accidental exposure to radiation can occur, for example, in an occupational setting such as a nuclear power plant, a facility that sterilizes articles or food by irradiation, or a hospital or cancer clinic. Accidental exposure to cytotoxic chemical agents can occur, for example, through industrial spills or emissions, or through unintentional contact with or inhalation or ingestion of such agents in the home or workplace, resulting in poisoning. Exposure can alternatively be intentional but malign, for example through criminal activity or hostile chemical or nuclear attack, including as an act of terrorism or war. Self-inflicted exposure for a non-medical reason, for example in a suicide attempt, can also occur.

However, exposure to radiation or cytotoxic chemical agents most commonly occurs intentionally in a medical setting, notably in treatment of certain serious or intractable diseases, especially cancer. Even where radiation is focused on a target tumor, exposure of healthy tissues in the vicinity of the tumor is generally unavoidable, despite best efforts to avoid such off-target exposure. Thus radiation therapy of cancers in the pelvic or abdominal region, for example cancers of the genitourinary system, often unavoidably results in concomitant exposure to radiation of mucosal tissues in the lower intestine, especially the descending colon and rectum. Likewise, radiation therapy of thoracic cancers can result in concomitant exposure to radiation of mucosal tissues in the esophagus, and radiation therapy of cancers in the head and neck region often unavoidably results in concomitant exposure to radiation of oropharyngeal mucosa. In the case of cytotoxic chemical agents administered as chemotherapeutics to cancer patients, systemic distribution in the body can deliver such agents to mucosal tissues throughout the body, including those in any or all parts of the GI tract, regardless of the location of the target cancer.

“Radiation” herein will be understood to include any form of radiation that interferes with, inhibits or disrupts normal processes of cell division in mucosal tissue exposed to such radiation. It includes higher frequency (shorter wavelength) electromagnetic radiation such as ultraviolet (UV) rays, X-rays and gamma-rays (γ-rays), as well as emanations of high-energy electrons or beta-particles (β-rays) and helium nuclei or alpha-particles (α-rays) from radionuclides. In general it is radiation carrying sufficient energy to be ionizing (i.e., to detach electrons from atoms or molecules) that is most likely to cause the kinds of adverse effects in the GI tract addressed by the present invention. X-rays administered for diagnostic imaging and related purposes, for example in the course of computed axial tomography (CAT) scans, have been implicated as a contributory cause of enteritis; however, the most common cause of radiation-induced GI adverse effects is radiation therapy for cancerous conditions. Radiation therapy can use any kind of ionizing radiation, most commonly γ-rays. Conditions other than cancer that can be treated by radiation therapy include trigeminal neuralgia and Graves' disease. Total body irradiation is used to prepare a patient for bone marrow transplant.

Ionizing radiation is cytotoxic in a number of ways, but principally through damage to the DNA of rapidly dividing cells. Such damage can occur by direct ionization, or indirectly through ionization of water molecules generating highly reactive free radicals, notably hydroxyl (OH) radicals.

As with any agent, cytotoxicity of radiation is dose-dependent. Absorbed radiation dose is normally measured in grays (Gy); 1 Gy represents absorption of 1 joule of radiation energy by 1 kg of matter. In radiation therapy, adverse effects are typically related to the dose administered over a course of therapy rather than the dose administered at any one time. The total dose of radiation administered to a target area of the body for cancer treatment is typically about 10 to about 100 Gy, more commonly about 20 to about 80 Gy. Such a dose is typically given in fractions of about 1 to about 3 Gy, more usually about 1.5 to about 2.5 Gy, e.g., about 1.8 to about 2 Gy, per day, for example 3-7 days per week (most commonly 5 days per week). A daily fraction can, if desired, be further fractionated.

A “course” of radiation therapy herein, when the dose is not fractionated, is one dose. When the dose is fractionated, a “course” of radiation therapy is a succession of fractional doses administered over a period of time, collectively providing a complete dose. Treatment may involve a single course or several courses of radiation therapy. Courses are usually separated by “rest periods” when no radiation is given.

Radiation doses absorbed from X-rays are much smaller than those occurring in radiation therapy. For example, a pelvic CAT scan results in a dose of about 25 mGy (0.025 Gy) and a diagnostic abdominal X-ray only about 1.5 mGy (0.0015 Gy).

Cytotoxic chemical agents include certain industrial chemicals that, if ingested, cause injury to the mucosal lining of the GI tract. However, of particular interest herein is GI injury associated with administration of cytotoxic chemotherapeutic drugs, including alkylating agents, platinum complexes, antimetabolites, antimitotic agents, topoisomerase inhibitors and intercalating agents.

Alkylating agents attach alkyl groups to DNA, resulting in cross-linking of guanine bases, in turn preventing uncoiling of the DNA double helix which is a necessary step in DNA replication and hence in cell division. Examples of alkylating agents include:
nitrogen mustards, e.g., bendamustine, canfosfamide, chlorambucil, chlornaphazine, cyclophosphamide, estramustine, glufosfamide, ifosfamide, mechlorethamine, melphalan, perfosfamide, prednimustine, trichlormethine, trofosfamide and uracil mustard;
nitrosoureas, e.g., carmustine, chlorozotocin, fotemustine, lomustine, nimustine ranimustine and streptozocin;
alkyl sulfonates, e.g., busulfan, improsulfan and piposulfan;
aziridines, e.g., carboquone, diaziquone and uredepa;
triethylenethiophosphoramide and related agents, e.g., altretamine, triethylene-melamine and triethylenephosphoramide; and
others not included above, e.g., dacarbazine, etoglucid, mitobronitol, mitolactol, pipobroman, procarbazine and temozolomide.

Platinum complexes coordinate to DNA and prevent repair and replication, thus have similar effects to alkylating agents (with which they are sometimes classified). Examples of platinum complexes include carboplatin, cisplatin, iproplatin, lobaplatin, nedaplatin, oxaliplatin, picoplatin, satraplatin and triplatin tetranitrate.

Antimetabolites interfere with DNA synthesis. Examples of antimetabolites include:
folic acid analogs and antagonists, e.g., denopterin, edatrexate, methotrexate, nolatrexed, pemetrexed, piritrexim, pteropterin, raltitrexed and trimetrexate;
purine analogs, e.g., azathioprine, cladibrine, clofarabine, fludarabine, 6-mercaptopurine, nelarabine, pentostatin, thiamiprine, thioguanine and tiazofurin; and
pyrimidine analogs, e.g., ancitabine, azacitidine, 6-azauridine, capecitabine, carmofur, cytarabine, decitabine, doxifluridine, enocitabine, floxuridine, fluorouracil, gemcitabine, tegafur and troxacitabine.

Antimitotic agents interfere with formation or assembly of microtubules necessary for mitosis (cell division). Antimitotic agents include:
colchicine;
vinca alkaloids, e.g., vinblastine, vincristine, vindesine, vinflunine and vinorelbine; and
taxanes, e.g., docetaxel, larotaxel, ortataxel, paclitaxel and tesetaxel.

Topoisomerase inhibitors are agents that interfere with the activity of enzymes involved in breakage and repair of the DNA backbone. Topoisomerase inhibitors include:
topoisomerase I inhibitors including camptothecin derivatives, e.g., 9-amino-camptothecin, belotecan, exatecan, irinotecan, rubitecan and topotecan; and
topoisomerase II inhibitors including podophyllum derivatives, e.g., etoposide and teniposide.

Intercalating agents intercalate between base pairs of DNA and may also have topoisomerase II inhibitory activity. Many exhibit antibiotic activity. Intercalating agents and other chemotherapeutic antibiotics include:
anthracyclines, e.g., aclacinomycin, amrubicin, carubicin, daunorubicin, doxorubicin, epirubicin, idarubicin, pirarubicin, valrubicin and zorubicin;
actinomycins, e.g., cactinomycin and dactinomycin; and
others not included above, e.g., bleomycin, mitomycin, peplomycin, plicamycin, porfiromycin, temsirolimus and zinostatin.

Appropriate doses of chemotherapeutic agents for anticancer therapy depend on the particular agent selected, the type, stage, location and aggressiveness of the cancer to be treated, the goal of treatment (e.g., palliative, curative, post-surgical, etc.), and other factors. Suitable doses can be identified based on published information on any individual agent. Because the anticancer effect depends on essentially the same mechanisms that are responsible for GI adverse effects, it will generally be difficult or impossible to separate therapeutic efficacy from such adverse effects by dose selection alone. A “therapeutically effective dose” of a cytotoxic chemotherapeutic agent herein is one that provides a benefit in one or more of reduction of tumor growth or size; slowing of tumor growth; reduction or slowing of tumor spread; or slowing, delaying or prevention of metastasis. Dose is usually calculated on the basis of a patient's body surface area (for example in mg/m²), particularly for administration by intravenous injection or infusion, as body surface area correlates with blood volume.

While most chemotherapeutic regimens involve intravenous administration, other routes of administration can be used for certain specific chemotherapeutics and in certain types of cancer. Such alternative routes include oral, buccal, sublingual, intranasal, intraocular, rectal, vaginal, transdermal and parenteral (other than intravenous, e.g., intradermal, subcutaneous, intramuscular, intra-arterial, intratracheal, intraventricular, intraperitoneal, etc.) routes, as well as inhalation and implantation.

Chemotherapy is generally not administered continuously for an indefinite period, but is given at short intervals (e.g., daily, every other day, twice weekly, weekly, every two weeks, etc.) for a treatment period, followed by a rest period when no chemotherapy is given. A complete treatment period followed by a rest period is known as a treatment cycle. In some situations, there may be only a single treatment in a cycle. A “course” of chemotherapy herein is the single dose or succession of doses within a treatment period.

The GI adverse effect moderated according to the present method can be induced by a single cytotoxic agent, for example a course of radiation therapy or a single cytotoxic chemotherapeutic agent, or by a combination of more than one such agent, for example a regimen of anticancer therapy comprising administering a plurality of chemotherapeutic agents, or radiation plus at least one chemotherapeutic agent.

A “subject” herein is a warm-blooded animal, generally a mammal such as, for example, a cat, dog, horse, cow, pig, mouse, rat or primate, including a human. In one embodiment the subject is human, for example a patient who has been or is scheduled within about 1 to about 30 days to be exposed to a cytotoxic agent, for example ionizing radiation and/or at least one cytotoxic chemotherapeutic agent. In a particular embodiment, the subject is a human cancer patient. Animal models in experimental investigations relevant to human disease are also examples of “subjects” herein, and can include for example rodents (e.g., mouse, rat, guinea pig), lagomorphs (e.g., rabbit), carnivores (e.g., cat, dog), or nonhuman primates (e.g., monkey, chimpanzee). Further, the subject can be an animal (for example a domestic, farm, working, sporting or zoo animal) in veterinary care.

The present method, in one embodiment, comprises administering to the subject a therapeutically effective amount of a compound selected from the group consisting of ORE1001 ((S,S)-2-[1-carboxy-2-[3-(3,5-dichlorobenzyl)-3H-imidazol-4-yl]-ethylamino]-4-methylpentanoic acid), pharmaceutically acceptable salts thereof and prodrugs thereof.

ORE1001 has in its chemical structure two acid moieties that, under suitable conditions, can form salts with suitable bases, and an amino group that, under suitable conditions, can form salts with suitable acids. Internal salts can also be formed. The compound can be used in its free acid form or in the form of an internal salt, an acid addition salt or a salt with a base.

Acid addition salts can illustratively be formed with inorganic acids such as mineral acids, for example sulfuric acid, phosphoric acids or hydrohalic (e.g., hydrochloric or hydrobromic) acids; with organic carboxylic acids such as (a) C₁₋₄ alkanecarboxylic acids which may be unsubstituted or substituted (e.g., halo-substituted), for example acetic acid, (b) saturated or unsaturated dicarboxylic acids, for example oxalic, malonic, succinic, maleic, fumaric, phthalic or terephthalic acids, (c) hydroxycarboxylic acids, for example ascorbic, glycolic, lactic, malic, tartaric or citric acids, (d) amino acids, for example aspartic or glutamic acids, or (e) benzoic acid; or with organic sulfonic acids such as C₁₋₄ alkanesulfonic acids or arylsulfonic acids which may be unsubstituted (e.g., halo-substituted), for example methanesulfonic acid or p-toluenesulfonic acid.

Salts with bases include metal salts such as alkali metal or alkaline earth metal salts, for example sodium, potassium or magnesium salts; or salts with ammonia or an organic amine such as morpholine, thiomorpholine, piperidine, pyrrolidine, a mono-, di- or tri-lower alkyl amine, for example ethylamine, tert-butylamine, diethylamine, diisopropylamine, triethylamine, tributylamine or dimethylpropylamine, or a mono-, di- or tri-(hydroxy lower alkyl) amine, for example monoethanolamine, diethanolamine or triethanolamine.

Alternatively, a prodrug of the compound or a salt of such prodrug can be used. A prodrug is a compound, typically itself having weak or no pharmaceutical activity, that is cleaved, metabolized or otherwise converted in the body of a subject to an active compound, in this case ORE1001. Examples of prodrugs are esters, amides, carbamates, carbonates, ketals, acetals, phosphates, phosphonates, sulfates and sulfonates. Various prodrugs of ORE1001, and methods of making such prodrugs, are disclosed, for instance, in above-referenced U.S. Pat. No. 6,632,830 and U.S. Published Patent Application No. 2004/0082496. Specific examples of prodrugs of ORE1001 include those wherein at least one of the carboxylic acid moieties is converted to a metabolically cleavable ester or amide such as a branched or unbranched alkyl ester (e.g., ethyl ester or isopropyl ester), branched or unbranched alkenyl ester, N-alkyl or N,N-dialkylaminoalkyl ester (e.g., dimethylaminoethyl ester), acylaminoalkyl ester, acyloxyalkyl ester (e.g., pivaloyloxymethyl ester), aryl ester (e.g., phenyl ester), arylalkyl ester (e.g., benzyl ester), alkylamide, dialkylamide or hydroxyamide. Alkyl and alkenyl groups in such esters and amides typically have up to 6 carbon atoms and are optionally substituted, for example with halo (e.g., fluoro or chloro), hydroxy or lower alkoxy (e.g., methoxy) substituents; aryl (e.g., phenyl) groups are optionally substituted, for example with lower alkyl (e.g., methyl), halo (e.g., fluoro or chloro), hydroxy or lower alkoxy (e.g., methoxy) substituents.

In a more particular embodiment, the compound administered to moderate a GI adverse effect of a cytotoxic agent is not a prodrug but is ORE1001 itself or a pharmaceutically acceptable salt thereof.

As previously noted, ORE1001 is the (S,S)-enantiomer of a compound having the formula

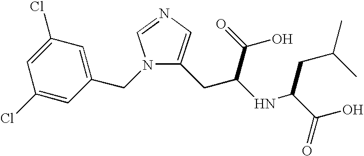

as disclosed for example by Dales et al. (2002), supra, together with a process for preparing such a compound. In brief, this process comprises treating (S)-histidine methyl ester with Boc₂O to provide a fully protected histidine derivative. The N-3 imidazole nitrogen is then selectively alkylated using the triflate of 3,5-dichlorobenzyl alcohol. Following Boc deprotection, reductive amination between the resulting alkylated histidine derivative and a β-ketoester furnishes a diester amine compound, which by hydrolysis yields 2-[1-carboxy-2-[3-(3,5-dichlorobenzyl)-3H-imidazol-4-yl]-ethylaminol-4-methylpentanoic acid as a mixture of diastereomers. The diastereomers can be separated and purified using HPLC and crystallization.

Other processes can be used to prepare ORE1001, including without limitation processes described in above-referenced U.S. Pat. No. 6,632,830 and U.S. Published Patent Application No. 2004/0082496.

The compound should be administered in an amount effective to moderate the GI adverse effect. What constitutes an effective amount depends on a number of factors, including the particular subject's age and body weight, the nature, location in the GI tract and severity of the adverse effect, the particular effect sought (e.g., reduction of inflammation, alleviation of symptoms, recovery from acute mucositis or enteritis, management of chronic mucositis or enteritis, etc.), the particular causal agent of the adverse effect and other factors. In the case of ORE1001, for most human subjects a dosage amount of about 0.5 to about 5000 mg/day, more typically about 10 to about 2400 mg/day, will be found suitable. In particular embodiments, the dosage employed is about 50 to about 2100 mg/day, or about 100 to about 2100 mg/day; illustratively about 50, about 100, about 200, about 300, about 400, about 500, about 600, about 700, about 800, about 900, about 1000, about 1100, about 1200, about 1300, about 1400, about 1500, about 1600, about 1700, about 1800, about 1900, about 2000 or about 2100 mg/day.

Where a salt or prodrug of ORE1001 is used, the amount administered should be an amount delivering a daily dosage of ORE1001 as set forth above.

The above dosages are given on a per diem basis but should not be interpreted as necessarily being administered on a once daily frequency. Indeed the compound, or salt or prodrug thereof, can be administered at any suitable frequency, for example as determined conventionally by a physician taking into account a number of factors, but typically about four times a day, three times a day, twice a day, once a day, every second day, twice a week, once a week, twice a month or once a month. The compound, or salt or prodrug thereof, can alternatively be administered more or less continuously, for example by parenteral infusion in a clinic or hospital setting. In some situations, a single dose may be administered, but more typically, administration is according to a regimen involving repeated dosage over a treatment period. In such a regimen the daily dosage and/or frequency of administration can, if desired, be varied over the course of the treatment period, for example introducing the subject to the compound at a relatively low dose and then increasing the dose in one or more steps until a full dose is reached.

The treatment period is generally as long as is needed to achieve a desired outcome, for example reduction of inflammation, alleviation of symptoms, recovery from acute mucositis or enteritis, management of chronic mucositis or enteritis, etc. In some situations, it will be found useful to administer the drug intermittently, for example for treatment periods of days, weeks or months separated by non-treatment periods. Such intermittent administration can be timed, for example, to correspond to courses of anticancer therapy with a cytotoxic agent.

In some embodiments, treatment with ORE1001 or a salt or prodrug thereof is administered to a subject who has already been exposed to a cytotoxic agent. Such a subject may already be suffering a GI adverse effect as a result of the exposure, or, if the exposure was very recent, may not yet display signs of such effect. Such embodiments of the invention are useful, for example, in post-exposure treatment of victims of accidental, malign or self-inflicted poisoning with a cytotoxic agent, as well as patients who have recently received radiation or chemotherapy.

In other embodiments, treatment with ORE1001 or a salt or prodrug thereof begins not later than the commencement of a course of treatment with a cytotoxic agent, for example 0 to about 30 days before the first dose of the cytotoxic agent in a course of therapy. Illustratively, treatment with ORE1001 or a salt or prodrug thereof can begin about 1 to about 30, for example about 1 to about 15, about 1 to about 10, about 1 to about 7 or about 1 to about 3 days prior to commencement of radiation or chemotherapy. There is some evidence that such anticipatory treatment is especially beneficial; see Example 5 herein. The ORE1001 (or salt or prodrug) treatment can continue for the duration of the course of treatment with the cytotoxic agent and beyond, for as long as needed.

Administration of the ORE1001 or salt or prodrug thereof can be by any suitable route, including without limitation oral, buccal, sublingual, intranasal, intraocular, rectal (e.g.. via enema), vaginal, transdermal or parenteral (e.g., intradermal, subcutaneous, intramuscular, intravenous, intra-arterial, intratracheal, intraventricular, intraperitoneal, etc.) routes, and including by inhalation or implantation. Route of administration of the ORE1001 or salt or prodrug thereof is independent of (i.e., can be the same as or different from) that of any cytotoxic agent with which it is concomitantly administered.

While it can be possible to administer the compound, or a salt or prodrug thereof unformulated as active pharmaceutical ingredient (API) alone, it will generally be found preferable to administer the API in a pharmaceutical composition that comprises the API and at least one pharmaceutically acceptable excipient. The excipient(s) collectively provide a vehicle or carrier for the API. Pharmaceutical compositions adapted for all possible routes of administration are well known in the art and can be prepared according to principles and procedures set forth in standard texts and handbooks such as those individually cited below.

USIP, ed. (2005) Remington: The Science and Practice of Pharmacy, 21st ed., Lippincott, Williams & Wilkins.

Allen et al. (2004) Ansel's Pharmaceutical Dosage Forms and Drug Delivery Systems, 8th ed., Lippincott, Williams & Wilkins.

Suitable excipients are described, for example, in Kibbe, ed. (2000) Handbook of Pharmaceutical Excipients, 3rd ed., American Pharmaceutical Association.

Examples of formulations that can be used as vehicles for delivery of the API in practice of the present invention include, without limitation, solutions, suspensions, powders, granules, tablets, capsules, pills, lozenges, chews, creams, ointments, gels, liposomal preparations, nanoparticulate preparations, injectable preparations, enemas, suppositories, inhalable powders, sprayable liquids, aerosols, patches, depots and implants.

Illustratively, in a liquid formulation suitable, for example, for parenteral, intranasal or oral delivery, the API can be present in solution or suspension, or in some other form of dispersion, in a liquid medium that comprises a diluent such as water. Additional excipients that can be present in such a formulation include a tonicifying agent, a buffer (e.g., a tris, phosphate, imidazole or bicarbonate buffer), a dispersing or suspending agent and/or a preservative. Such a formulation can contain micro- or nanoparticulates, micelles and/or liposomes. A parenteral formulation can be prepared in dry reconstitutable form, requiring addition of a liquid carrier such as water or saline prior to administration by injection.

For rectal delivery, the API can be present in dispersed form in a suitable liquid (e.g., as an enema), semi-solid (e.g., as a cream or ointment) or solid (e.g., as a suppository) medium. The medium can be hydrophilic or lipophilic.

For oral delivery, the API can be formulated in liquid or solid form, for example as a solid unit dosage form such as a tablet or capsule. Such a dosage form typically comprises as excipients one or more pharmaceutically acceptable diluents, binding agents, disintegrants, wetting agents and/or antifrictional agents (lubricants, anti-adherents and/or glidants). Many excipients have two or more functions in a pharmaceutical composition. Characterization herein of a particular excipient as having a certain function, e.g., diluent, binding agent, disintegrant, etc., should not be read as limiting to that function.

Suitable diluents illustratively include, either individually or in combination, lactose, including anhydrous lactose and lactose monohydrate; lactitol; maltitol; mannitol; sorbitol; xylitol; dextrose and dextrose monohydrate; fructose; sucrose and sucrose-based diluents such as compressible sugar, confectioner's sugar and sugar spheres; maltose; inositol; hydrolyzed cereal solids; starches (e.g., corn starch, wheat starch, rice starch, potato starch, tapioca starch, etc.), starch components such as amylose and dextrates, and modified or processed starches such as pregelatinized starch; dextrins; celluloses including powdered cellulose, microcrystalline cellulose, silicified microcrystalline cellulose, food grade sources of α- and amorphous cellulose and powdered cellulose, and cellulose acetate; calcium salts including calcium carbonate, tribasic calcium phosphate, dibasic calcium phosphate dihydrate, monobasic calcium sulfate monohydrate, calcium sulfate and granular calcium lactate trihydrate; magnesium carbonate; magnesium oxide; bentonite; kaolin; sodium chloride; and the like. Such diluents, if present, typically constitute in total about 5% to about 99%, for example about 10% to about 85%, or about 20% to about 80%, by weight of the composition. The diluent or diluents selected preferably exhibit suitable flow properties and, where tablets are desired, compressibility.

Lactose, microcrystalline cellulose and starch, either individually or in combination, are particularly useful diluents.

Binding agents or adhesives are useful excipients, particularly where the composition is in the form of a tablet. Such binding agents and adhesives should impart sufficient cohesion to the blend being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; glucose; polydextrose; starch including pregelatinized starch; gelatin; modified celluloses including methylcellulose, carmellose sodium, hydroxypropylmethylcellulose (HPMC or hypromello se), hydroxypropyl-cellulose, hydroxyethylcellulose and ethylcellulose; dextrins including maltodextrin; zein; alginic acid and salts of alginic acid, for example sodium alginate; magnesium aluminum silicate; bentonite; polyethylene glycol (PEG); polyethylene oxide; guar gum; polysaccharide acids; polyvinylpyrrolidone (povidone), for example povidone K-15, K-30 and K-29/32; polyacrylic acids (carbomers); polymethacrylates; and the like. One or more binding agents and/or adhesives, if present, typically constitute in total about 0.5% to about 25%, for example about 0.75% to about 15%, or about 1% to about 10%, by weight of the composition.

Povidone is a particularly useful binding agent for tablet formulations, and, if present, typically constitutes about 0.5% to about 15%, for example about 1% to about 10%, or about 2% to about 8%, by weight of the composition.

Suitable disintegrants include, either individually or in combination, starches including pregelatinized starch and sodium starch glycolate; clays; magnesium aluminum silicate; cellulose-based disintegrants such as powdered cellulose, microcrystalline cellulose, methylcellulose, low-substituted hydroxyprop ylcellulo se, carmellose, carmellose calcium, carmellose sodium and croscarmellose sodium; alginates; povidone; crospovidone; polacrilin potassium; gums such as agar, guar, locust bean, karaya, pectin and tragacanth gums; colloidal silicon dioxide; and the like. One or more disintegrants, if present, typically constitute in total about 0.2% to about 30%, for example about 0.2% to about 10%, or about 0.2% to about 5%, by weight of the composition.

Croscarmellose sodium and crospovidone, either individually or in combination, are particularly useful disintegrants for tablet or capsule formulations, and, if present, typically constitute in total about 0.2% to about 10%, for example about 0.5% to about 7%, or about 1% to about 5%, by weight of the composition.

Wetting agents, if present, are normally selected to maintain the drug or drugs in close association with water, a condition that is believed to improve bioavailability of the composition. Non-limiting examples of surfactants that can be used as wetting agents include, either individually or in combination, quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers (polyoxyethylene and polyoxypropylene block copolymers); polyoxyethylene fatty acid glycerides and oils, for example polyoxyethylene (8) caprylic/capric mono- and diglycerides, polyoxyethylene (35) castor oil and polyoxyethylene (40) hydrogenated castor oil; polyoxyethylene alkyl ethers, for example ceteth-10, laureth-4, laureth-23, oleth-2, oleth-10, oleth-20, steareth-2, steareth-10, steareth-20, steareth-100 and polyoxyethylene (20) cetostearyl ether; polyoxyethylene fatty acid esters, for example polyoxyethylene (20) stearate, polyoxyethylene (40) stearate and polyoxyethylene (100) stearate; sorbitan esters; polyoxyethylene sorbitan esters, for example polysorbate 20 and polysorbate 80; propylene glycol fatty acid esters, for example propylene glycol laurate; sodium lauryl sulfate; fatty acids and salts thereof, for example oleic acid, sodium oleate and triethanolamine oleate; glyceryl fatty acid esters, for example glyceryl monooleate, glyceryl monostearate and glyceryl palmitostearate; sorbitan esters, for example sorbitan monolaurate, sorbitan monooleate, sorbitan monopalmitate and sorbitan monostearate; tyloxapol; and the like. One or more wetting agents, if present, typically constitute in total about 0.25% to about 15%, preferably about 0.4% to about 10%, and more preferably about 0.5% to about 5%, by weight of the composition.

Wetting agents that are anionic surfactants are particularly useful. Illustratively, sodium lauryl sulfate, if present, typically constitutes about 0.25% to about 7%, for example about 0.4% to about 4%, or about 0.5% to about 2%, by weight of the composition.

Lubricants reduce friction between a tableting mixture and tableting equipment during compression of tablet formulations. Suitable lubricants include, either individually or in combination, glyceryl behenate; stearic acid and salts thereof, including magnesium, calcium and sodium stearates; hydrogenated vegetable oils; glyceryl palmitostearate; talc; waxes; sodium benzoate; sodium acetate; sodium fumarate; sodium stearyl fumarate; PEGs (e.g., PEG 4000 and PEG 6000); poloxamers; polyvinyl alcohol; sodium oleate; sodium lauryl sulfate; magnesium lauryl sulfate; and the like. One or more lubricants, if present, typically constitute in total about 0.05% to about 10%, for example about 0.1% to about 8%, or about 0.2% to about 5%, by weight of the composition. Magnesium stearate is a particularly useful lubricant.

Anti-adherents reduce sticking of a tablet formulation to equipment surfaces. Suitable anti-adherents include, either individually or in combination, talc, colloidal silicon dioxide, starch, DL-leucine, sodium lauryl sulfate and metallic stearates. One or more anti-adherents, if present, typically constitute in total about 0.1% to about 10%, for example about 0.1% to about 5%, or about 0.1% to about 2%, by weight of the composition.

Glidants improve flow properties and reduce static in a tableting mixture. Suitable glidants include, either individually or in combination, colloidal silicon dioxide, starch, powdered cellulose, sodium lauryl sulfate, magnesium trisilicate and metallic stearates. One or more glidants, if present, typically constitute in total about 0.1% to about 10%, for example about 0.1% to about 5%, or about 0.1% to about 2%, by weight of the composition.

Talc and colloidal silicon dioxide, either individually or in combination, are particularly useful anti-adherents and glidants.

Other excipients such as buffering agents, stabilizers, antioxidants, antimicrobials, colorants, flavors and sweeteners are known in the pharmaceutical art and can be used. Tablets can be uncoated or can comprise a core that is coated, for example with a nonfunctional film or a release-modifying or enteric coating. Capsules can have hard or soft shells comprising, for example, gelatin and/or HPMC, optionally together with one or more plasticizers.

A pharmaceutical composition useful herein typically contains ORE1001 or a salt or prodrug thereof in an amount of about 1% to about 99%, more typically about 5% to about 90% or about 10% to about 60%, by weight of the composition. A unit dosage form such as a tablet or capsule can conveniently contain an amount of the compound providing a single dose, although where the dose required is large, it may be necessary or desirable to administer a plurality of dosage forms as a single dose. Illustratively, a unit dosage form can comprise the compound in an amount of about 10 to about 1000 mg, for example about 50 to about 900 mg or about 100 to about 800 mg; or, in particular illustrative instances, about 50, about 100, about 150, about 200, about 250, about 300, about 350, about 400, about 450, about 500, about 550, about 600, about 650, about 700, about 750 or about 800 mg.

ORE1001 is an ACE2 inhibitor; however, its mode of action is not a limitation to aspects of the invention described above. It is believed, without such limitation and without being bound by theory, that inhibition of ACE2 is a component of the mechanism of action of ORE1001 in reducing GI adverse effects of cytotoxic agents. Other ACE2 inhibitors are known in the art, and it is contemplated according to some embodiments of the invention that any such inhibitor can be used in place of ORE1001, with adjustment of dose and other modalities of administration as necessary. A suitable dose for any ACE2 inhibitor will generally but without limitation be found in the range given above for ORE1001.

Thus in one embodiment, a method for moderating a GI adverse effect induced by exposure to at least one cytotoxic agent in a subject comprises administering to the subject a therapeutically effective amount of an ACE2 inhibitor.

Any ACE2 inhibitor can be used. In general, it will be found useful to select an ACE2 inhibitor having relatively high affinity for ACE2, as expressed for example by IC₅₀ or Ki, whether measured in vitro or in vivo. In one embodiment, the ACE2 inhibitor selected is one that exhibits in vitro an ACE2 IC₅₀ and/or an ACE2 K, not greater than about 1000 nM, for example not greater than about 500 nM, not greater than about 250 nM, or not greater than about 100 nM.

ACE2 inhibitors are known to differ not only in their affinity for ACE2 but also in their selectivity for binding to ACE2 as opposed to the more ubiquitous ACE. In one embodiment, the ACE2 inhibitor exhibits selectivity for ACE2 versus ACE, as expressed by the ratio of IC₅₀(ACE) to IC₅₀(ACE2), of at least about 10², for example at least about 10³, or at least about 10⁴.

Peptide and non-peptide ACE2 inhibitors can be used. Examples of peptide ACE2 inhibitors, and methods for preparing them, can be found for example in above-cited U.S. Pat. No. 6,900,033, which is incorporated herein by reference in its entirety. Peptide compounds exhibiting relatively strong inhibition of ACE2 illustratively include those having peptide sequences identified as DX-512, DX-513, DX-524, DX-525, DX-529, DX-531, DX-599, DX-600, DX-601 and DX-602 in U.S. Pat. No. 6,900,033. Antibodies that bind specifically to the ACE2 protein and thereby inhibit ACE2 activity can also be used in methods and compositions of the present invention.

For many purposes, it will be found preferable to use a non-peptide or “small molecule” ACE2 inhibitor. Such compounds tend to be easier to prepare, especially on a large or commercial scale, have lower cost, and present fewer problems in administration and delivery to the active site in the body. In various embodiments, therefore, the ACE2 inhibitor comprises a non-peptide compound or a pharmaceutically acceptable salt thereof or a prodrug thereof.

Illustratively, an ACE2 inhibitor can be of a type disclosed generically in above-cited U.S. Pat. No. 6,632,830, which is incorporated herein by reference in its entirety, including any of the specific compounds disclosed therein along with methods of preparation thereof. In one embodiment, the non-peptide compound comprises a zinc coordinating moiety and an amino acid mimicking moiety.

More specifically, the non-peptide compound can have the formula

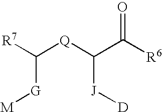

as disclosed in U.S. Pat. No. 6,632,830, wherein
R⁶ is hydroxyl or a protecting prodrug moiety;
R⁷ is hydrogen, carboxylic acid, ether, alkoxy, an amide, a protecting prodrug moiety, hydroxyl, thiol, heterocyclyl, alkyl or amine;
Q is CH₂, O, NH or NR³, wherein R³ is substituted or unsubstituted C₁₋₅ branched or straight chain alkyl, C₂₋₅ branched or straight chain alkenyl, substituted or unsubstituted acyl, aryl or a C₃₋₈ ring;
G is a covalent bond or a CH₂, ether, thioether, amine or carbonyl linking moiety;
M is heteroaryl, substituted with at least one subanchor moiety comprising a substituted or unsubstituted cycloalkyl or aryl ring, linked thereto through a sublinking moiety (CH₂)ₙ or (CH₂)ₙO(CH₂)ₙ where n is an integer from 0 to 3;
J is a bond or a substituted or unsubstituted alkyl, alkenyl or alkynyl moiety; and
D is alkyl, alkenyl, alkynyl, aryl or heteroaryl, optionally linked to G or M to form a ring.

In one embodiment, in the above formula for the non-peptide compound, R⁶ is hydroxyl, R⁷ is carboxylic acid, Q is NH and G is CH₂.

In one embodiment, in the above formula for the non-peptide compound, the heteroaryl group of M is imidazolyl, thienyl, triazolyl, pyrazolyl or thiazolyl. Independently of the selection of heteroaryl group, the subanchor moiety according to this embodiment is C₃₋₆ cycloalkyl, phenyl, methylenedioxyphenyl, naphthalenyl, or phenyl having 1 to 3 substituents independently selected from halo, C₁₋₆ alkyl, C₃₋₆ cycloalkyl, trifluoromethyl, C₁₋₆ alkoxy, trifluoromethoxy, phenyl, cyano, nitro and carboxylic acid groups, and is linked to the heteroaryl group through a (CH₂)ₙ or (CH₂)O(CH₂) sublinking moiety, where n is an integer from 0 to 3.

In one embodiment, in the above formula for the non-peptide compound, J is a bond or CH₂ moiety and D is C_{1 6} alkyl, C_{3 6} cycloalkyl or phenyl.

In a more particular embodiment, in the formula for the non-peptide compound:
R⁶ is hydroxyl;
R⁷ is carboxylic acid;
Q is NH;
G is CH₂;
M is imidazolyl, thienyl, triazolyl, pyrazolyl or thiazolyl, linked through a (CH₂)ₙ or (CH₂)O(CH₂) sublinking moiety, where n is an integer from 0 to 3, to a subanchor moiety that is C₃₋₆ cycloalkyl, phenyl, methylenedioxyphenyl, naphthalenyl, or phenyl having 1 to 3 substituents independently selected from halo, C₁₋₆ alkyl, C₃₋₆ cycloalkyl, trifluoromethyl, C₁₋₆ alkoxy, trifluoromethoxy, phenyl, cyano, nitro and carboxylic acid groups;
J is a bond or CH₂ moiety; and
D is C₁₋₆ alkyl, C₃₋₆ cycloalkyl or phenyl.

According to any of the above embodiments the non-peptide compound can be present in any enantiomeric configuration, e.g., (R,R), (R,S), (S,R) or (S,S), or as a mixture, for example a racemic mixture, of enantiomers. However, in general it is found preferable that the compound be present in the (S,S)-configuration. In one embodiment, the compound is in the (S,S)-configuration and is substantially enantiomerically pure. For example, the compound can exhibit an enantiomeric purity of at least about 90%, at least about 95%, at least about 98% or at least about 99%, by weight of all enantiomeric forms of the compound present.

Illustrative compounds specifically disclosed in U.S. Pat. No. 6,632,830 include the following, each of which can be in any enantiomeric form, illustratively in the (S,S)-configuration:
2-[1-carboxy-2-[3-(4-trifluoromethylbenzyl)-3H-imidazol-4-yl]ethylamino]-4-methyl-pentanoic acid;
2-[1-carboxy-2-[3-naphthalen-1-ylmethyl-3H-imidazol-4-yl]ethylamino]-4-methyl-pentanoic acid;
2-[1-carboxy-2-[3-(4-chlorobenzyl)-3H-imidazol-4-yl]ethylamino]-4-methylpentanoic acid;
2-[1-carboxy-2-[3-(3,4-dichlorobenzyl)-3H-imidazol-4-yl]ethylamino]-4-methyl-pentanoic acid;
2-[1-carboxy-2-[3-(4-cyanobenzyl)-3H-imidazol-4-yl]ethylamino]-4-methylpentanoic acid;
2-[1-carboxy-2-[3-(3-chlorobenzyl)-3H-imidazol-4-yl]ethylamino]-4-methylpentanoic acid;
2-[1-carboxy-2-[3-(3,5-dichlorobenzyl)-3H-imidazol-4-yl]ethylamino]-4-methyl-pentanoic acid;
2-[1-carboxy-2-[3-(4-methylbenzyl)-3H-imidazol-4-yl]ethylamino]-4-methylpentanoic acid;
2-[1-carboxy-2-[3-(3,4-dimethylbenzyl)-3H-imidazol-4-yl]ethylamino]-4-methyl-pentanoic acid;
2-[1-carboxy-2-[3-(3-methylbenzyl)-3H-imidazol-4-yl]ethylamino]-4-methylpentanoic acid;
2-[1-carboxy-2-[3-(3,5-dimethylbenzyl)-3H-imidazol-4-yl]ethylamino]-4-methyl-pentanoic acid;
2-[1-carboxy-2-[3-(4-trifluoromethoxybenzyl)-3H-imidazol-4-yl]ethylamino]-4-methyl-pentanoic acid;
2-[1-carboxy-2-[3-(4-isopropylbenzyl)-3H-imidazol-4-yl]ethylamino]-4-methyl-pentanoic acid;
2-[1-carboxy-2-[3-(4-tert-butylbenzyl)-3H-imidazol-4-yl]ethylamino]-4-methylpentanoic acid;
2-[1-carboxy-2-[3-(4-nitrobenzyl)-3H-imidazol-4-yl]ethylamino]-4-methylpentanoic acid;
2-[1-carboxy-2-[3-(2,3-dimethoxybenzyl)-3H-imidazol-4-yl]ethylamino]-4-methyl-pentanoic acid;
2-[1-carboxy-2-[3-(2,3-difluorobenzyl)-3H-imidazol-4-yl]ethylamino]-4-methyl-pentanoic acid;
2-[1-carboxy-2-[3-(2,3-dichlorobenzyl)-3H-imidazol-4-yl]ethylamino]-4-methyl-pentanoic acid;
2-[1-carboxy-2-[3-(3-trifluoromethylbenzyl)-3H-imidazol-4-yl]ethylamino]-4-methyl-pentanoic acid;
2-[2-(3-benzo[1,3]dioxol-5-ylmethyl-3H-imidazol-4-yl)-1-carboxyethylamino]-4-methylpentanoic acid;
2-[1-carboxy-2-[3-(2-cyclohexylethyl)-3H-imidazol-4-yl]ethylamino]-4-methyl-pentanoic acid;
2-[1-carboxy-2-[3-phenethyl-3H-imidazol-4-yl]ethylamino]-4-methylpentanoic acid;
2-[1-carboxy-2-[3-(3-iodobenzyl)-3H-imidazol-4-yl]ethylamino]-4-methylpentanoic acid;
2-[1-carboxy-2-[3-(3-fluorobenzyl)-3H-imidazol-4-yl]ethylamino]-4-methylpentanoic acid;
2-[1-carboxy-2-[3-benzyloxymethyl-3H-imidazol-4-yl]ethylamino]-4-methylpentanoic acid;
2-[1-carboxy-2-[3-(4-butylbenzyl)-3H-imidazol-4-yl]ethylamino]-4-methylpentanoic acid;
2-[1-carboxy-2-[3-(2-methylbenzyl)-3H-imidazol-4-yl]ethylaminol-4-methylpentanoic acid;
2-[1-carboxy-2-[2-phenylthiazol-4-yl]ethylamino]-4-methylpentanoic acid;
2-[1-carboxy-2-[1-benzyl)-1H-pyrazol-4-yl]ethylaminol-4-methylpentanoic acid; and
2-[1-carboxy-2-[3-(2-methylbiphenyl-3-ylmethyl)-3H-imidazol-4-yl]ethylamino]-4-methylpentanoic acid.

As in the case of ORE1001 described above, any of the above compounds can be present in the above form or in the form of a pharmaceutically acceptable salt thereof, or a prodrug thereof.

Additional compounds having ACE2 inhibitory activity that can be used in practice of the present invention have been disclosed by Huentelman et al. (2004), supra, including NAAE (N-(2-aminoethyl)-1-aziridineethanamine).

Further additional compounds having ACE2 inhibitory activity that can be used in practice of the present invention have been disclosed by Rella et al. (2006) J. Chem. Inf. Model. 46(2):708-716. This publication discloses structure-based pharmacophore design and virtual screening for novel ACE2 inhibitors, including 17 compounds that are reported to display an inhibitory effect on ACE2 activity, the six most active exhibiting IC₅₀ values in the range of 62-179 μM.

Modalities of administration of the ACE2 inhibitor, including optional use of salt or prodrug forms of the compound, formulation, route and frequency of administration, and timing of administration relative to exposure to a cytotoxic agent, are as set out for ORE1001 above.

In some embodiments, the GI adverse effect moderated comprises one or more of oral, pharyngeal or esophageal mucositis or enteritis (including colitis and/or proctitis). For example, the GI adverse effect can comprise oral or oropharyngeal mucositis resulting from radiation therapy to the head and neck region or from systemic chemotherapy. Alternatively or in addition, the GI adverse effect can comprise esophageal mucositis resulting from radiation therapy to the thoracic region or from systemic chemotherapy. Alternatively or in addition, the GI adverse effect can comprise enteritis, colitis or proctitis resulting from radiation therapy to the pelvic or abdominal region or from systemic chemotherapy.

In some embodiments the moderating of a GI adverse effect by administration of ORE1001 or a salt or prodrug thereof, or by administration of an ACE2 inhibitor, comprises reduction of severity of one or more of oral, pharyngeal or esophageal mucositis, colitis or proctitis.

In some embodiments the moderating of a GI adverse effect by administration of ORE1001 or a salt or prodrug thereof, or by administration of an ACE2 inhibitor, comprises reduction of one or more of inflammation, edema or necrosis.

In some embodiments the moderating of a GI adverse effect by administration of ORE1001 or a salt or prodrug thereof, or by administration of an ACE2 inhibitor, comprises amelioration of symptoms of the adverse effect such as xerostomia, difficulty in eating, swallowing or speaking, pain or diarrhea.

In some embodiments the moderating of a GI adverse effect by administration of ORE1001 or a salt or prodrug thereof, or by administration of an ACE2 inhibitor, comprises amelioration of a secondary outcome of the adverse effect such as poor nutrition, weight loss, infective disease, dental or periodontal disease, or a psychiatric condition such as depression.

A particular benefit of the present invention accrues to patients having cancer or other disease in which therapy with a cytotoxic agent such as ionizing radiation or a chemotherapeutic agent is indicated, though the moderation of GI adverse effects of such therapy as described above. Because, in some cases, a patient's tolerance of such therapy is limited by GI adverse effects, the moderation of these effects opens up the possibility of such therapy to patients who otherwise could not receive it. Likewise, patients who otherwise could tolerate only low, marginally effective doses of radiation or chemotherapeutics can receive higher, more effective doses when concomitantly given ORE1001 or a salt or prodrug thereof, or an ACE2 inhibitor, in accordance with various embodiments of the present invention.

Anticancer therapy with one or more cytotoxic agents, in combination with a further agent to moderate adverse side effects of the cytotoxic agents, is known in the art as adjunct therapy. The present invention provides, in an important embodiment, a new adjunct therapy method.

According to one aspect of this embodiment, a method for treating a cancerous condition in a subject comprises administering to the subject (a) at least one cytotoxic anticancer agent and (b) a compound selected from the group consisting of ORE1001, pharmaceutically acceptable salts thereof and prodrugs thereof in an amount effective to moderate a GI adverse effect induced by the anticancer agent.

According to another aspect of this embodiment, a method for treating a cancerous condition in a subject comprises administering to the subject (a) at least one cytotoxic anticancer agent and (b) an ACE2 inhibitor in an amount effective to moderate a GI adverse effect induced by the anticancer agent.

The at least one cytotoxic anticancer agent can comprise ionizing radiation, alone or as part of a regimen further comprising one or more chemotherapeutic agents. Any dose of radiation, for example as disclosed hereinabove, carrying a risk of GI adverse effects to non-target tissues in the vicinity of the target tumor can be used. Illustratively a radiation dose of about 20 to about 80 Gy, in fractions of about 1 to about 3 Gy, is administered in adjunct therapy that further comprises administration of ORE1001 or a salt or prodrug thereof, or an ACE2 inhibitor.

The at least one cytotoxic anticancer agent can comprise at least one chemotherapeutic agent, optionally together with radiation. Any chemotherapeutic agent, for example as disclosed hereinabove, or combination thereof can be used, in a therapeutically effective dose as defined herein. Illustratively the at least one chemotherapeutic agent can be selected from the group consisting of alkylating agents (including nitrogen mustards, nitrosoureas, alkyl sulfonates, aziridines, triethylenethiophosphoramide and related agents, and others), platinum complexes, antimetabolites (including folic acid analogs and antagonists, purine analogs and pyrimidine analogs), antimitotic agents (including colchicine, vinca alkaloids and taxanes), topoisomerase inhibitors (including topoisomerase I inhibitors and topoisomerase II inhibitors), intercalating agents (including anthracyclines, actinomycins and others), and combinations thereof.

In certain embodiments the at least one chemotherapeutic agent is selected from the group consisting of bendamustine, canfosfamide, chlorambucil, chlornaphazine, cyclophosphamide, estramustine, glufosfamide, ifosfamide, mechlorethamine, melphalan, perfosfamide, prednimustine, trichlormethine, trofosfamide, uracil mustard, carmustine, chlorozotocin, fotemustine, lomustine, nimustine, ranimustine, streptozocin, busulfan, improsulfan, piposulfan, carboquone, diaziquone, uredepa, triethylenethiophosphoramide, altretamine, triethylenemelamine, triethylenephosphoramide, dacarbazine, etoglucid, mitobronitol, mitolactol, pipobroman, procarbazine, temozolomide, carboplatin, cisplatin, iproplatin, lobaplatin, nedaplatin, oxaliplatin, picoplatin, satraplatin, triplatin tetranitrate, denopterin, edatrexate, methotrexate, nolatrexed, pemetrexed, piritrexim, pteropterin, raltitrexed, trimetrexate, azathioprine, cladibrine, clofarabine, fludarabine, 6-mercaptopurine, nelarabine, pentostatin, thiamiprine, thioguanine, tiazofurin, ancitabine, azacitidine, 6-azauridine, capecitabine, carmofur, cytarabine, decitabine, doxifluridine, enocitabine, floxuridine, fluorouracil, gemcitabine, tegafur, troxacitabine, colchicine, vinblastine, vincristine, vindesine, vinflunine, vinorelbine, docetaxel, larotaxel, ortataxel, paclitaxel, tesetaxel, 9-aminocamptothecin, belotecan, exatecan, irinotecan, rubitecan, topotecan, etoposide, teniposide, aclacinomycin, amrubicin, carubicin, daunorubicin, doxorubicin, epirubicin, idarubicin, pirarubicin, valrubicin, zorubicin, cactinomycin, dactinomycin, bleomycin, mitomycin, peplomycin, plicamycin, porfiromycin, temsirolimus, zinostatin and combinations thereof.

The present method is appropriate for treatment of any kind of cancerous condition. In one embodiment, the condition comprises one or more solid tumors, which can be primary or secondary (metastatic). Solid tumors include, for example, adenocarcinomas, carcinomas, hemangiomas, liposarcomas, melanomas and sarcomas. Alternatively or in addition, the condition can comprise nonsolid tumors such as cancers of the blood and lymphatic systems, including leukemia (acute and chronic forms) and lymphoma. The cancerous condition can occur in any organ or body part including, without limitation, the anus, bile duct, bone, bone marrow, brain, breast, cervix, colon, duodenum, esophagus, gallbladder, head and neck, ileum, jejunum, kidney, larynx, liver, lung, mouth, ovary, pancreas, pelvis, penis, pituitary, prostate, rectum, skin, stomach, testes, thyroid, urinary bladder, uterus and vagina.

More particularly, the cancerous condition can comprise one or more of the following: acinar adenocarcinoma, acinar carcinoma, acral-lentiginous melanoma, actinic keratosis, adenocarcinoma, adenocystic carcinoma, adenosquamous carcinoma, adnexal carcinoma, adrenal rest tumor, adrenocortical carcinoma, adult T-cell lymphoma, aldosterone-secreting carcinoma, alveolar soft part sarcoma, amelanotic melanoma, ameloblastic carcinoma, ampullary carcinoma, anal canal cancer, anaplastic thyroid carcinoma, angiosarcoma, apocrine carcinoma, Askin's tumor, astrocytoma, basal cell carcinoma, basaloid carcinoma, basophilic leukemia, basosquamous cell carcinoma, B-cell lymphoma, biliary cancer, bone cancer, bone marrow cancer, botryoid sarcoma, brain cancer, breast cancer, bronchioalveolar carcinoma, bronchogenic adenocarcinoma, bronchogenic carcinoma, Burkitt-like leukemia, Burkitt's lymphoma, carcinoid, carcinoma en cuirasse, carcinoma ex pleomorphic adenoma, cervical cancer, chloroma, cholangiocellular carcinoma, chondrosarcoma, choriocarcinoma, choroid plexus carcinoma, clear cell adenocarcinoma, colon cancer, colorectal cancer, comedocarcinoma, cortisol-producing carcinoma, cylindrical cell carcinoma, dedifferentiated liposarcoma, diffuse lymphoma, ductal adenocarcinoma of the prostate, ductal carcinoma, ductal carcinoma in situ, duodenal cancer, eccrine carcinoma, embryonal carcinoma, endometrial carcinoma, endometrial stromal sarcoma, endometrioid adenocarcinoma, endometrioid carcinoma, epithelioid sarcoma, esophageal cancer, Ewing's sarcoma, exophytic carcinoma, fibroblastic sarcoma, fibrocarcinoma, fibrolamellar carcinoma, fibrosarcoma, follicular lymphoma, follicular thyroid carcinoma, gallbladder cancer, gastric adenocarcinoma, giant cell carcinoma, giant cell sarcoma, giant cell tumor of bone, granulocytic leukemia, granulosa cell carcinoma, hairy cell leukemia, head and neck cancer, hemangioma, hemangiosarcoma, hepatocellular carcinoma, Hodgkin's disease, Hiirthle cell carcinoma, ileal cancer, infiltrating lobular carcinoma, inflammatory carcinoma of the breast, intraductal carcinoma, intraepidermal carcinoma, jejunal cancer, Kaposi's sarcoma, Krukenberg's tumor, Kulchitsky cell carcinoma, Kupffer cell sarcoma, large cell carcinoma, large cell lymphoma, larynx cancer, Lennert's lymphoma, lentigo maligna melanoma, leukemia, leukemia cutis, liposarcoma, liver cancer, lobular carcinoma, lobular carcinoma in situ, lung cancer, lymphoblastic leukemia, lymphoblastic lymphoma, lymphoepithelioma, lymphoma cutis, lymphosarcoma, malignant melanoma, mantle cell lymphoma, marginal zone lymphoma, mast cell leukemia, medullary carcinoma, medullary thyroid carcinoma, megakaryoblastic leukemia, meningeal carcinoma, Merkel cell carcinoma, micropapillary carcinoma, mixed cell sarcoma, monocytic leukemia, mucinous carcinoma, mucoepidermoid carcinoma, mucosal melanoma, myeloblastic leukemia, myelogenous leukemia, myelomonocytic leukemia, myxoid liposarcoma, myxosarcoma, nasopharyngeal carcinoma, nodular melanoma, non-clear cell renal cancer, non-Hodgkin's lymphoma, non-small cell lung cancer, oat cell carcinoma, ocular melanoma, oral cancer, osteoid carcinoma, osteosarcoma, ovarian cancer, Paget's carcinoma, pancreatic cancer, papillary adenocarcinoma, papillary carcinoma, papillary thyroid carcinoma, pelvic cancer, periampullary carcinoma, phyllodes tumor, pituitary cancer, plasmacytic leukemia, pleomorphic liposarcoma, preinvasive carcinoma, primary intraosseous carcinoma, prostate cancer, rectal cancer, renal cell carcinoma, rhabdomyosarcoma, Rieder cell leukemia, round cell liposarcoma, scar cancer, schistosomal bladder cancer, schneiderian carcinoma, sebaceous carcinoma, signet-ring cell carcinoma, skin cancer, small cell lung cancer, small cell osteosarcoma, small cleaved cell lymphoma, small noncleaved cell lymphoma, soft tissue sarcoma, spindle cell carcinoma, spindle cell sarcoma, squamous cell carcinoma, stomach cancer, superficial spreading melanoma, synovial sarcoma, T-cell lymphoma, telangiectatic sarcoma, terminal duct carcinoma, testicular cancer, thyroid cancer, transitional cell carcinoma, tubular carcinoma, tumorigenic melanoma, undefined lymphoma, undifferentiated carcinoma, undifferentiated leukemia, urachal adenocarcinoma, urinary bladder cancer, uterine cancer, uterine corpus carcinoma, uveal melanoma, vaginal cancer, verrucous carcinoma, villous carcinoma, well-differentiated liposarcoma, Wilm's tumor and yolk sac tumor.

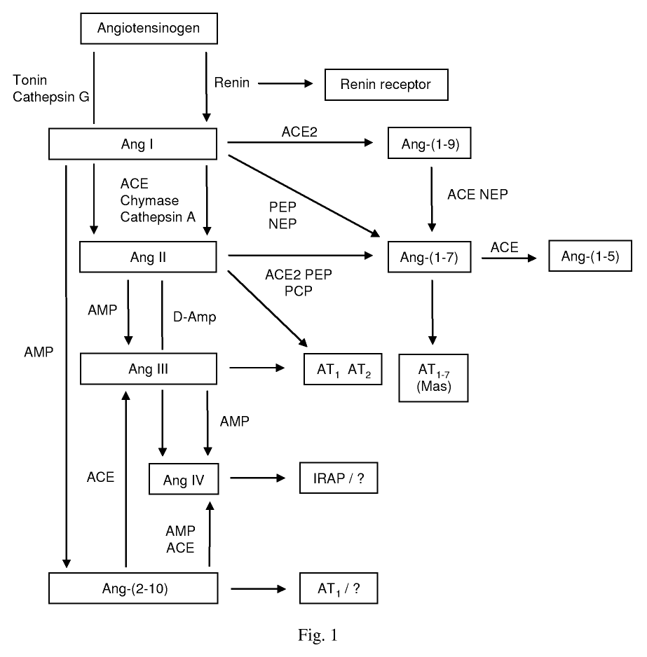

In accordance with methods of the invention, ORE1001 has been found to inhibit TNFα-induced activation of NF-κB in recombinant HeLa reporter cells. This finding is reported in greater detail in Example 2 below. ORE1001 is a known ACE2 inhibitor, thus its effect on the renin-angiotensin system (RAS) might be predicted to involve increase in level of angiotensin II (see FIG. 1), which as indicated above is implicated in a variety of pro-inflammatory effects. Contrary to such prediction, it is now shown that activation of NF-κB, a key mediator for synthesis of pro-inflammatory cytokines, is not promoted but inhibited by ORE1001.

ORE1001 has further been found to inhibit in vivo basal NF-κB-dependent transcription in recombinant reporter mice. This finding is reported in greater detail in Example 3 below, and appears to further support an anti-inflammatory effect of ORE1001 that is contrary to expectation based on its ACE2 inhibitory properties and present understanding of the role of ACE2 in the RAS.

It has now been surprisingly found that in a rat model for radiation proctitis, administration of ORE1001 reduced severity and histopathological indicators of proctitis including inflammation and necrosis. This result, reported in greater detail in Example 5 below, is strong evidence suggesting therapeutic effectiveness of ORE1001 in moderating GI adverse effects of cytotoxic agents in human patients.

A composition of matter that comprises two or more interacting therapeutic agents, whether for administration together or separately, is known herein as a “therapeutic combination”. Thus agents administered in adjunct therapy for treatment of a cancerous condition, as contemplated herein, are an example of a therapeutic combination. Two or more active agents administered in adjunct therapy can be formulated in one pharmaceutical preparation (single dosage form) for administration to the subject at the same time, or in two or more distinct preparations (separate dosage forms) for administration to the subject at the same or different times, e.g., sequentially. The two distinct preparations can be formulated for administration by the same route or by different routes.

Separate dosage forms can optionally be co-packaged, for example in a single container or in a plurality of containers within a single outer package, or co-presented in separate packaging (“common presentation”). As an example of co-packaging or common presentation, a kit is contemplated comprising, in a first container, a chemotherapeutic agent and, in a second container, ORE1001 or a salt or prodrug thereof, or an ACE2 inhibitor. In another example, a chemotherapeutic agent and ORE1001 or a salt or prodrug thereof, or an ACE2 inhibitor, are separately packaged and available for sale independently of one another, but are co-marketed or co-promoted for use according to the invention. The separate dosage forms may also be presented to a subject separately and independently, for use according to the invention.

In one aspect, the invention provides a therapeutic combination comprising (a) at least one cytotoxic anticancer agent and (b) a compound selected from the group consisting of ORE1001, pharmaceutically acceptable salts thereof and prodrugs thereof in an amount effective, when administered to a subject receiving the anticancer agent, to moderate a GI adverse effect induced in the subject by the anticancer agent. The at least one cytotoxic anticancer agent can be, for example, a chemotherapeutic agent or combination of such agents as listed illustratively above.

In another aspect, the invention provides a therapeutic combination comprising (a) at least one cytotoxic anticancer agent and (b) an ACE2 inhibitor in an amount effective, when administered to a subject receiving the anticancer agent, to moderate a GI adverse effect induced in the subject by the anticancer agent. Again, the at least one cytotoxic anticancer agent can be, for example, a chemotherapeutic agent or combination of such agents as listed illustratively above.

### Examples

### Example 1

### ACE2 mRNA Expression in Normal and Disease States

Donoghue et al. (2000), supra, reported finding ACE2 transcripts mainly in heart, kidney and testis, out of 23 normal human tissues examined, and ACE2 protein (via immunohistochemistry) predominantly in the endothelium of coronary and intrarenal vessels and in renal tubular epithelium.

Further, Tipnis et al. (2000) J. Biol. Chem. 275(43):33238-33243 reported Northern blotting analyses showing that the ACE2 mRNA transcript is most highly expressed in testis, kidney and heart.

Komatsu et al. (2002) DNA Seq. 13:217-220 reported molecular cloning of mouse angiotensin-converting enzyme-related carboxypeptidase (mACE2) showing 83% identity with human ACE2, and Northern blot analysis showing transcripts were expressed mainly in kidney and lungs.

More recently, Gembardt et al. (2005) Peptides 26:1270-1277 analyzed ACE2 mRNA and protein expression in various normal tissues of mice and rats, reporting at least detectable levels of ACE2 mRNA in all tested organs of both species (ventricle, kidney, lung, liver, testis, gallbladder, forebrain, spleen, thymus, stomach, ileum, colon, brainstem, atrium, and adipose tissue). In both species, ileum tissue showed the highest expression of ACE2 mRNA, with the mouse exceeding the rat in ACE2 mRNA expression in this organ and also in the kidney and colon.

Burrel et al. (2005) Eur. Heart J. 26:369-375 recently reported that myocardial infarction increases ACE2 expression in rat and human heart.

ACE2 mRNA expression has now been examined in various human tissues from normal and diseased subjects, using the BioExpress® System of Gene Logic Inc. This system includes mRNA expression data from about 18,000 samples, of which about 90% are from human tissues, comprising both normal and diseased samples from about 435 disease states. In brief, human tissue samples, either from surgical biopsy or post-mortem removal, were processed for mRNA expression profile analysis using Affymetrix GeneChips®. Each tissue sample was examined by a board-certified pathologist to confirm pathological diagnoses. RNA isolation, cDNA synthesis, cRNA amplification and labeling, hybridizations, and signal normalization were carried out using standard Affymetrix protocols. Computational analysis was performed using Genesis Enterprise System® Software and the Ascenta® software system (Gene Logic Inc).

In agreement with Donoghue et al. (2000), supra, and Tipnis et al. (2000), supra, the present study showed relatively high levels of ACE2 transcripts in normal human heart, kidney and testis (data not shown). However, excluding those three normal tissues, the top 8 highest expression levels of ACE2 mRNA in 70 additional normal human tissues that were examined are listed in Table 1 below, in descending order of mean expression level (given as the “average relative level,” i.e., sample set signal level in arbitrary units, normalized to the lowest signal level in all tested samples, averaged for two different probe fragments).

These top 8 normal tissues in Table 1 (and heart, kidney and testis as well) showed average relative levels of ACE2 mRNA expression greater than 4.0, while the remaining 62 normal tissues examined showed average relative levels less than 4.0.

Table 1 also shows that four of the top five highest expression levels of ACE2 mRNA in normal human tissues (other than heart, kidney and testis) were in components of the gastrointestinal tract, namely (in descending order of expression level): duodenum, small intestine, colon and stomach.

TABLE 1
Relative levels of ACE2 mRNA expression in normal tissues

| Sample Set | Average Relative Level |
|---|---|
| Duodenum | 221.2 |
| Small Intestine | 167.9 |
| Gallbladder | 109.9 |
| Colon | 13.6 |
| Stomach | 10.1 |
| Ovary | 5.7 |
| Pancreas | 4.3 |
| Liver | 4.2 |

Examination of ACE2 mRNA expression in disease states encompassed by the BioExpress® System showed elevation of ACE2 mRNA in only a few conditions, mainly inflammatory conditions of components of the gastrointestinal tract. Thus, Table 2 shows that ACE2 mRNA expression was elevated (in descending order of average fold change vs. normal) in inflammatory conditions of the stomach (chronic gastritis), major salivary gland (excluding parotid) (chronic sialadenitis), and colon (Crohn's disease, active (chronic or acute inflammation)). In contrast, the levels of ACE2 mRNA in colon with active ulcerative colitis (chronic or acute inflammation), and in small intestine with active Crohn's disease (chronic or acute inflammation), were substantially unchanged from the already significant levels in corresponding normal tissues shown in Table 1.

TABLE 2
Effects of inflammatory conditions on ACE2 mRNA expression in
digestive tract tissues

| | Average |
|---|---|
| | Fold Change |
| Sample Set | vs. Normal |
| Stomach, Chronic Gastritis | 8.2 |
| Major Salivary Gland (Excluding Parotid), Chronic | 7.5 |
| Sialadenitis | |
| Colon, Crohn's Disease, Active (Chronic Inflammation) | 2.2 |
| Colon, Crohn's Disease, Active (Acute Inflammation) | 1.7 |
| Colon, Ulcerative Colitis, Active (Chronic Inflammation) | 0.9 |
| Colon, Ulcerative Colitis, Active (Acute Inflammation) | 1.0 |
| Small Intestine, Crohn's Disease, Active (Chronic | 0.4 |
| Inflammation) | |
| Small Intestine, Crohn's Disease, Active | 0.8 |
| (Acute Inflammation) | |

The above findings taken together show that 4 of the top 11 highest expression levels of ACE2 mRNA found in normal human tissues are in components of the digestive tract, and that the majority of examined disease conditions that involve elevated ACE2 mRNA expression are inflammatory conditions of the digestive tract. Accordingly, these findings suggest that high levels of ACE2 mRNA expression could be a pathogenic factor and, hence, reduction of ACE2 activity could provide therapeutic benefit, in at least some inflammatory conditions of the digestive tract, particularly in the stomach (chronic gastritis), major salivary gland (chronic sialadenitis), and colon (Crohn's disease with chronic or acute inflammation). Further, although ACE2 mRNA levels were not elevated in colon with ulcerative colitis or small intestine with Crohn' s disease, the already substantial levels of such mRNA in normal colon and small intestine suggest at least that ACE2 activity is present and, therefore, could still constitute a pathogenic factor in these two diseased tissues.

### Example 2

### Inhibition by ORE1001 of TNFα-Induced Activation of NF-κB in Recombinant HeLa Reporter Cells

Both in human IBD and in murine models of IBD, inflammation is likely to depend, at least in part, on activation and nuclear translocation of NF-κB family members. See, e.g., Fichtner-Feigl et al. (2005) J. Clin. Invest. 115:3057-3071 and sources cited therein. Thus, in Th1-mediated inflammations dependent on IL-12 and/or IL-23, synthesis of these cytokines is regulated by NF-κB transcription factors. In Th2-mediated inflammations dependent on IL-4 or IL-13, synthesis of these cytokines is also dependent on NF-κB transcription factors, albeit more indirectly than that of IL-12 and IL-23. Thus one method of treating the inflammation of IBD can be to administer agents that inhibit NF-κB activity, and indeed Fichtner-Feigl et al. (2005), supra, have shown that NF-κB decoy oligodeoxynucleotides (ODNs) that prevent NF-κB activation of gene expression are effective in treating and preventing various models of Th1- and Th2-mediated IBD in mice, including acute trinitrobenzene sulfonic acid- (TNBS-) induced colitis, as assessed by clinical course and effect on Th1 cytokine production; chronic TNBS-induced colitis, inhibiting both production of IL-23/IL-17 and development of fibrosis; and oxazolone-induced colitis, a Th2-mediated inflammatory process.

To test the ACE2 inhibitor ORE1001 for anti-inflammatory activity relevant to IBD, effects of the compound on activation of NF-κB-dependent transcription by TNFα were examined in recombinant reporter cells containing a construct with a luciferase reporter gene under control of NF-κB-dependent regulatory sequences, thereby allowing detection of NF-κB-dependent transcription by measuring reporter enzyme using a conventional luciferase activity assay based on detection of generated light.

In particular, HeLa cells (American Type Culture Collection) were grown in Dulbecco's modified Eagles medium (DMEM) supplemented with 10% fetal calf serum and transiently transfected with an NF-κB—luc construct (Stratagene, Inc.), as follows (with all incubation steps at 37 C unless otherwise indicated). Cells were seeded and grown to about 70% confluency in a 10 cm cell culture dish. Plasmid DNA (10 μg) was added to 1 ml serum free DMEM media in a tube. Fugene 6 transfection reagent (30 μl) (Roche) was then pipetted slowly into the tube and the contents were gently mixed by inversion. The mixture was incubated at room temperature for 15 minutes and then added dropwise to cells in one 10 cm dish. Following incubation for 24 hours, cells were detached from the plate with Trypsin-EDTA (Gibco-BRL), transferred to wells in a clear-bottom white 96-well test plate (Fisher) with 100 μl per well serum free DMEM, at a density of 3×10⁴ cells per well, and allowed to attach overnight. Compound (ORE1001) was then added to wells at a concentration of approximately 0, 0.008, 0.04, 0.2, 1.0 or 5.0 μM, followed immediately by addition of TNFα (R&D) to a final concentration of 20 ng/ml. After incubation for 6 hours, 100 μl of Bright-Glo Luciferase buffer (Promega, Cat#E2610) was added, and the plate was incubated at room temperature, with mild shaking, for 10 min. Bioluminescence was then measured using a Veritas luminometer (Turner BioSystems). Each plotted data point represents the average bioluminescence of 4 independent wells.

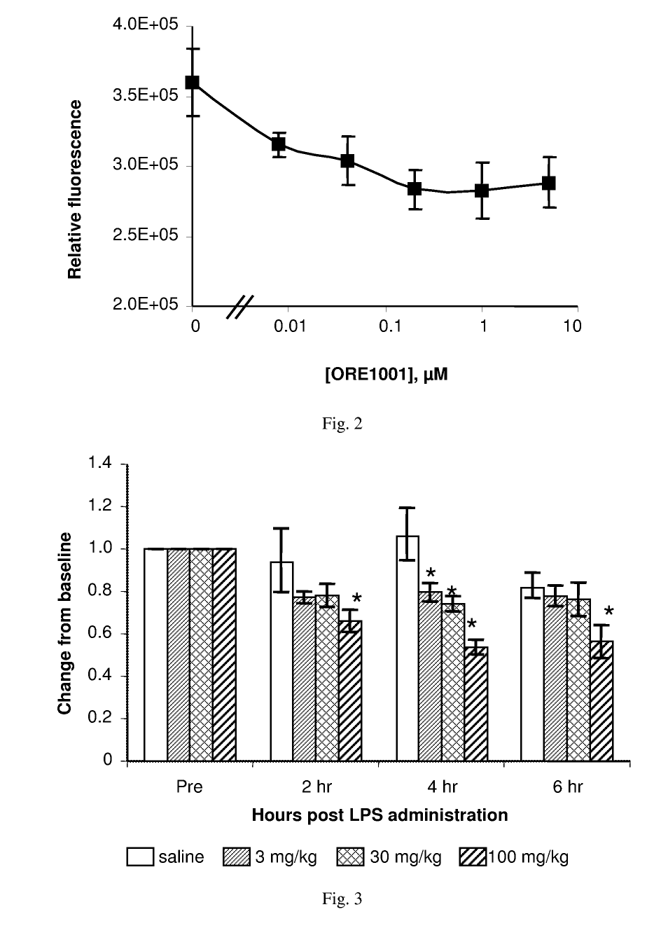

As shown in FIG. 2, ORE1001 significantly inhibited TNFα-induced activation of NF-κB-dependent transcription at all tested concentrations, with over 80% inhibition at 8 nM and maximal inhibition over 95% at 0.2 μM. These results indicate that the ACE2 inhibitor ORE1001 has potent anti-inflammatory activity, namely inhibition of activation of the NF-κB signaling pathway by the inflammatory cytokine TNFα, that is relevant to IBD. The present inventors are not aware of any previous report of such anti-inflammatory activity for any ACE2 inhibitor.

### Example 3

### Inhibition by ORE1001 of in Vivo Basal NF-κB-Dependent Transcription in Recombinant Reporter Mice

ORE1001 was further tested for in vivo anti-inflammatory activity by examining its effects on basal levels of NF-κB-dependent transcription in mice engineered in the germline with a construct containing an NF-κB enhancer linked to a luciferase gene (i.e., NF-κB::Luc mice), such that this NF-κB reporter construct is present in all cells of the mice.

More particularly, transgenic NF-κB::Luc mice were generated using three NF-κB response elements from the Igic light chain promoter fused to a firefly luciferase gene as described by Carlsen et al. (2002) J. Immunol. 168:1441-1446. Pronuclear microinjection of purified construct DNA was used to generate transgenic founders in the C57BL/6 XCBA/J background. Founders were subsequently back crossed to the C57BL/6 albino background. All experimental protocols were approved by the Institutional Animal Care and Use Committee and conform to the ILAR guide for the care and used of laboratory animals. For in vivo imaging, NF-κB::Luc mice were injected intraperitoneally with luciferin (150 mg/kg) 10 minutes before imaging, anesthetized (using 1-3% isoflurane) and placed into a light-tight camera box. Mice were imaged for up to two minutes from the dorsal or ventral aspects at high-resolution settings with a field of view of 20 cm. The light emitted from the transgene was detected by an IVIS® Imaging System 200 Series (Xenogen Corporation, Alameda, Calif.), digitized and displayed on a monitor. The Living Image® software (Xenogen Corporation, Alameda, Calif.; see Rice et al. (2002) J. Biomed. Opt. 6:432-440) displays data from the camera using a pseudocolor scale with colors representing variations of signal intensity. Signal data were also quantitated and archived using the Living Image® software. Photons of light were quantitated using an oval region of interest (ROI) of varying sizes depending on the procedure, as described further below.

For luciferase assays, organs were extracted and snap frozen in liquid nitrogen. All tissue samples were placed in lysis buffer with inhibitors (Passive Lysis Buffer (Promega) and Complete Mini Protease Inhibitor Cocktail (Roche, Indianapolis, Ind.)), and were homogenized using a tissue homogenizer (Handishear, Hand-held homogenizer, VirTis, Gardiner, N.Y.). Tissue homogenates were centrifuged and clarified lysates were used for luminometer assays and western blots. For the luminometer assays, Luciferase Assay Substrate (Luciferase Assay System, Promega) was prepared as indicated by the manufacturer and placed in disposable cuvettes. Tissue homogenates (20 μl) and substrate (100 82 l) were mixed and measurements were taken in a Veritas Microplate Luminometer (Turner Designs, Sunnyvale, Calif.) with the parameters of a 2 second delay, 10-second. Background luminescence readings were obtained and the background readings were subtracted from the luminescent data. Protein concentrations were determined using the BCA Protein Assay Kit (Pierce, Rockford, Ill.) following the manufacturer's protocols and analyzed using a VERSAmax tunable microplate reader and associated Softmax Pro version 3.1.2 software (Molecular Devices, Sunnyvale Calif.). The luminescence for each of the protein lysates was calculated as arbitrary units of light per microgram of protein. Statistical analyses include MEAN, SEM and ANOVA and students t-test between treatment groups.

To test for in vivo effects of ORE1001 on basal levels of NF-κB-dependent transcription, male NF-κB::Luc mice were subjected to quantitative in vivo imaging of the abdominal area (using a fixed ROI of 2.76×3.7 cm) as described above, immediately before, and at 2, 4 and 6 hours after subcutaneous administration of 0, 3, 30 or 100 mg/kg ORE1001 in saline. Whole body imaging showed that ORE1001 significantly inhibited basal in vivo levels of NF-κB-dependent transcription of the luciferase reporter gene, primarily in the abdominal region. As shown by the quantitative imaging data in FIG. 3, at 4 hours post LPS administration ORE1001 significantly inhibited basal in vivo levels of NF-κB-dependent transcription in the selected abdominal ROI by over 40% at 300 mg/kg (p<0.01 by ANOVA and Student's t-test), and to lesser but still significant extents at both lower doses.

In contrast to the results observed in NF-κB::Luc mice, no significant effect of ORE1001 was observed on basal in vivo levels of reporter luciferase expression in AP-1::Luc mice constructed similarly to the present NF-κB::Luc mice (data not shown), in which reporter transcription was driven by an enhancer element responsive to activator protein-1 (AP-1), a known protooncogene thought to be involved in cell proliferation and tumor promotion.

### Example 4

### ORE1001 Inhibits in Vivo LPS-Induced NF-κB-Dependent Transcription in Recombinant Reporter Mice

Bacterial lipopolysaccharide (LPS), a major component of the cell wall of gram-negative bacteria, is a highly biologically active molecule that stimulates macrophages to produce and release TNFα. See, e.g., Jersmann et al. (2001) Infection and Immunity 69(3):1273-1279, and sources cited therein. One of the recognized associations of bacterial infection with cardiovascular events is the activation of endothelium and up-regulation of adhesion molecules. The two major proinflammatory mediators implicated in the causation of cardiovascular events, bacterial LPS and TNFα have been found to cooperate to enhance the adhesive properties of endothelial cells by synergistically increasing expression of human endothelial adhesion molecules through activation of NF-κB and p38 mitogen-activated protein kinase signaling pathways.

ORE1001 was further tested for in vivo anti-inflammatory activity by examining its effects on bacterial LPS-induced NF-κB-dependent transcription, in NF-κB::Luc mice. In particular, inflammation was induced in these mice at 6-10 weeks of age by administration of 0.5 mg/kg (i.v.) soluble LPS (sLPS; Sigma) one hour after administration of ORE1001. Mice were subjected to quantitative abdominal imaging at 2, 4 and 6 h following LPS administration, as described above. In confirmatory experiments, and at the time point with the greatest modulation of luciferase signal, animals were euthanized and tissues were collected and preserved for further analysis. Luciferase signal was quantitated from several regions of interest. Statistical analyses include MEAN, SEM and ANOVA and student t-test between treatment groups.

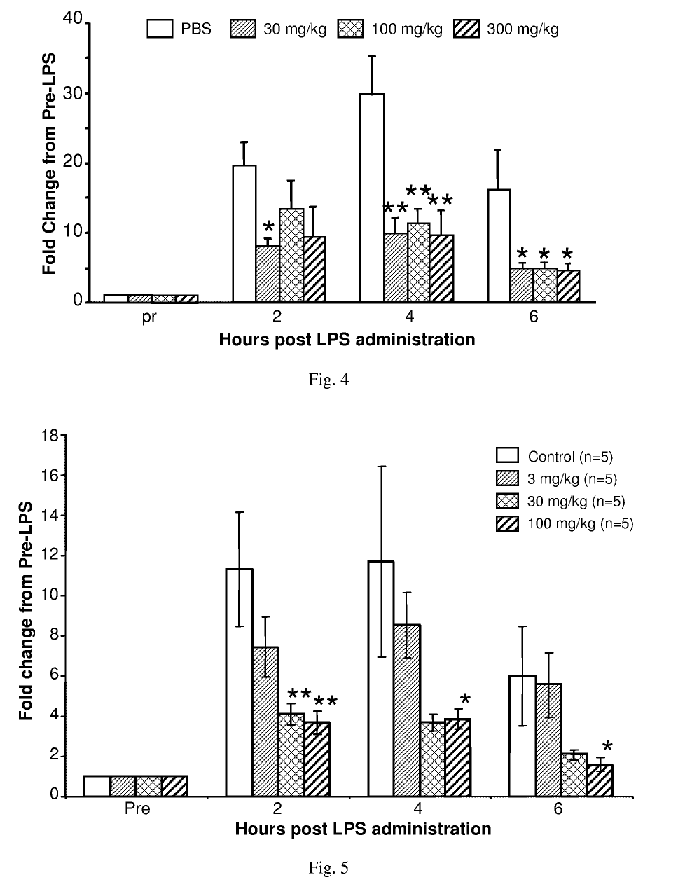

Whole body imaging showed that ORE1001 significantly inhibited LPS-induced in vivo levels of NF-κB-dependent transcription of the luciferase reporter gene, again primarily in the abdominal region. As shown by the quantitative imaging data in FIG. 4, LPS induced a strong NF-κB-dependent luciferase signal in control mice, indicating a strong NF-κB signaling response, as expected. In contrast, mice that were pretreated with ORE1001 showed a significantly reduced LPS-induced NF-κB signaling response, which could be measured quantitatively in the abdominal region. As inhibition of NF-κB-dependent luciferase activity was observed over the entire dose range of ORE1001 in this experiment (30 mg/kg, 100 mg/kg, 300 mg/kg), the experiment was repeated using a slightly lower dose range (3-100 mg/kg). As shown in FIG. 5, in this lower dose range, ORE1001 significantly reduced LPS-induced NF-κB signaling at 30 and 100 mg/kg. These results show that systemic (subcutaneous) administration of the ACE2 inhibitor ORE1001 showed significant in vivo anti-inflammatory activity, predominantly in the abdominal region, against bacterial LPS-induced NF-κB-dependent transcription as well as against basal NF-κB-dependent transcription.

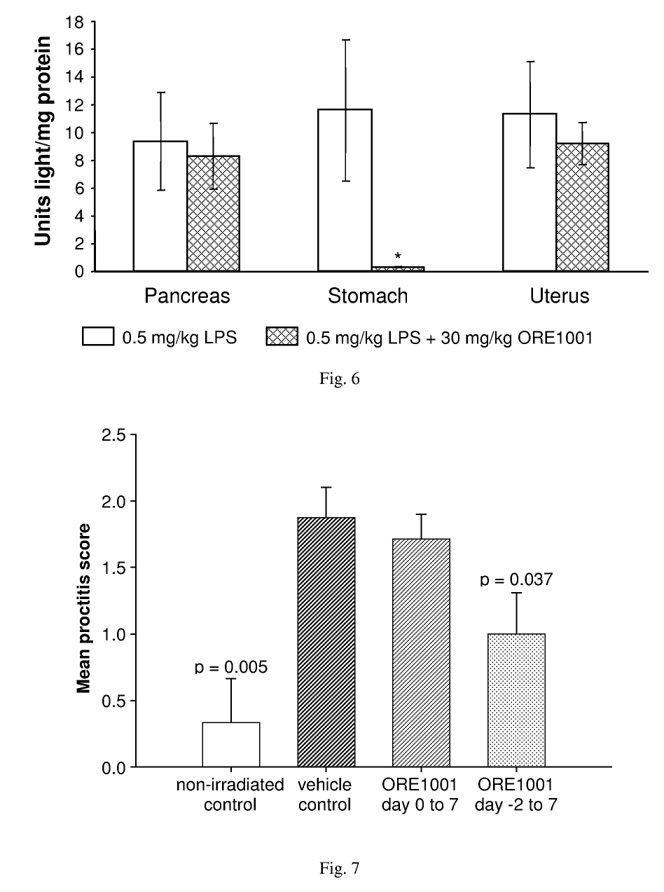

Examination of selected organs extracted from NF-κB::Luc mice treated with 0.5 mg/kg LPS and ORE1001 at 30 mg/kg or with 0.5 mg/kg LPS alone (FIG. 6) showed a significant (about 37-fold) reduction of LPS-induced NF-κB-dependent transcription in stomachs of ORE1001 treated mice, compared to mice treated with LPS alone, but no statistically significant decrease in LPS-induced NF-κB signaling in pancreas and uterus, or in any other organ or organ part that was analyzed (data not shown), namely, liver, kidney, spleen, small intestine, large intestine (colon), mesenteric lymph nodes, cecum (first part of the colon after the small intestine), ovary, uterus, submandibular lymph nodes, brain, heart and lung.

ORE1001 inhibition of LPS-induced NF-κB activity in the mouse stomach is consistent with the present observation (above) of ACE2 mRNA expression in normal stomach tissue of human subjects, and with the report of ACE2 mRNA expression in the mouse stomach by Gembardt et al. (2005), supra. The fact that no inhibition of LPS-induced NF-κB activity was observed in other murine tissues previously reported to express high levels of ACE2 mRNA (e.g., kidney, small intestine or colon; see Gembardt et al. (2005), supra) shows that the inhibitory effect on LPS-induced NF-κB signaling predominantly in the abdominal region following systemic (subcutaneous) administration of ORE1001 is primarily due to some activity of this ACE2 inhibitor in the stomach.

### Example 5

### ORE1001 Reduces Severity and Histopathological Indicators of Radiation-Induced Proctitis in Rats

A rat model was used to determine whether ORE1001 can reduce severity of radiation-induced proctitis, as assessed by endoscopy, and can reduce histopathological indicators of radiation-induced proctitis. This is a validated model, as described for example in the publications cited individually below and incorporated herein by reference.

Northway et al. (1988) Cancer 62:1962-1969.

Kang et al. (2000) J. Korean Med. Sci. 15:682-689.

Female Lewis rats of body weight 175-200 g and 6-8 weeks old were randomized into 3 groups of 8 animals each, for induction of proctitis by radiation as described below. In addition, a group of 8 female Lewis rats of body weight 175-200 g and 6-8 weeks old were followed as untreated, non-irradiated controls. Animals were acclimated prior to study commencement. The study was performed in animal rooms provided with filtered air at a temperature of 70±5° F. (˜21±3° C.) and relative humidity of 50±20%, and a 12 h/12 h light/dark cycle. Animals were fed with LabDiet 5053 sterile irradiated rat chow. Water was provided ad libitum.

Proctitis was induced by a single 17.5 Gy dose of radiation at a rate of 1 Gy/minute administered to each animal on day 0. Lead shielding was used to cover the animal except for a 3×4 cm area of the lower pelvis, permitting exposure to radiation of an approximately 2 cm length of rectum and distal descending colon. Radiation was generated with a Kimtron 160 kV, 15 mA source at a focal distance of 30 cm, hardened with a 0.35 mm Cu filtration system. Prior to irradiation, animals were anesthetized with an intraperitoneal injection of ketamine (100 mg/kg) and xylazine (5 mg/kg). Animals were inclined in a slightly head-down position, to allow gravity to move non-target abdominal organs out of the radiation path as much as possible.

ORE1001 in a vehicle of 0.5% carboxymethylcellulose (CMC) in sterile water was administered per os at a daily dose of 600 mg/kg in 5 ml/kg vehicle from day 0 to day 7 (first group) or from day −2 to day 7 (second group). A third group received vehicle only, from day −2 to day 7.

All animals were weighed daily and examined for diarrhea and fecal blood. On day 8, all animals were examined by rodent endoscope under isoflurane anesthesia and were scored for severity of proctitis. Following endoscopy, animals were euthanized by CO₂ inhalation and the descending colon and rectum to the anus were taken as a single tissue sample for histological examination. Histopathology was recorded by scoring inflammation, edema and necrosis.

Endoscopy data are summarized in FIG. 7. Irradiation resulted in a major increase in proctitis severity (compare vehicle control with non-irradiated control). ORE1001 administered from day 0 to day 7 gave a minor but statistically non-significant reduction in proctitis severity. When ORE1001 was administered from day −2 to day 7, i.e., with administration beginning 2 days before irradiation, a substantial reduction in proctitis severity was obtained.

No major differences in body weight change were seen among the treatments in this study.

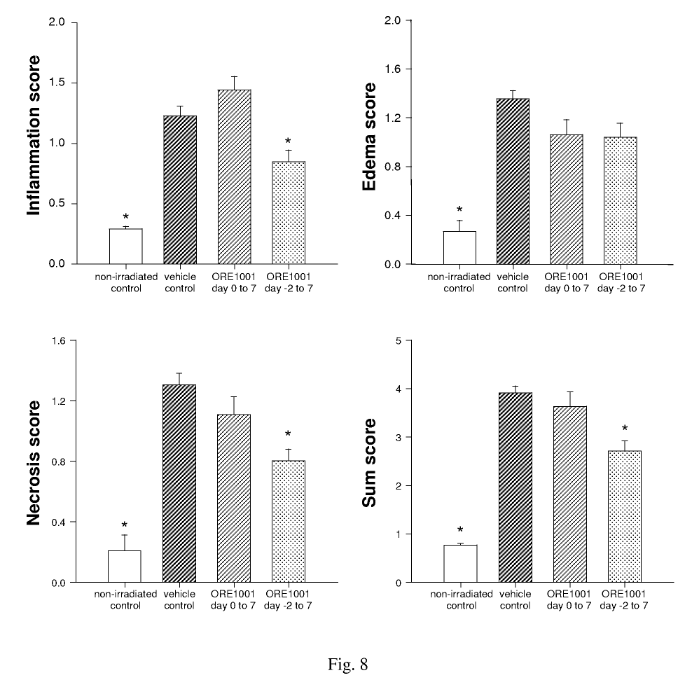

Histopathology data are summarized in FIG. 8 and substantially conform to the endoscopy results. ORE1001 administered from day −2 to day 7 gave statistically significant reductions at least in inflammation and necrosis scores, and in the sum of inflammation, edema and necrosis scores. ORE1001 administered from day 0 to day 7 showed a statistically non-significant trend for reduction in edema, necrosis and sum scores.

These results provide the first indication that the ACE2 inhibitor ORE1001 can ameliorate GI adverse effects of radiation.

All patents and publications cited herein are incorporated by reference into this application in their entirety.

The words “comprise”, “comprises”, and “comprising” are to be interpreted inclusively rather than exclusively.

What is claimed is:

1. A method for moderating a gastrointestinal adverse effect induced by exposure to at least one cytotoxic agent in a subject, comprising administering to the subject a therapeutically effective amount of an ACE2 inhibitor.

2. The method of claim 1, wherein the at least one cytotoxic agent comprises ionizing radiation and/or at least one cytotoxic chemical agent.

3. The method of claim 1, wherein the gastrointestinal adverse effect occurs in one or more of the mouth, pharynx, esophagus, stomach, duodenum, jejunum, ileum, cecum, colon and rectum.

4. The method of claim 1, wherein the gastrointestinal adverse effect comprises enteritis, colitis, proctitis and/or oral mucositis.

5. The method of claim 1, wherein the subject is a cancer patient, and said exposure results from a course of anticancer therapy with radiation and/or at least one cytotoxic chemotherapeutic agent administered to the patient.

6. The method of claim 5, wherein administration of the ACE2 inhibitor begins before the first dose of radiation or chemotherapeutic agent in the course of anticancer therapy.

7. The method of claim 6, wherein administration of the ACE2 inhibitor begins about 1 to about 30 days before said first dose of radiation or chemotherapeutic agent.

8. The method of claim 1, wherein the ACE2 inhibitor is administered to the subject in an amount of about 0.5 to about 5000 mg/day.

9. The method of claim 1, wherein the ACE2 inhibitor exhibits in vitro an ACE2 IC₅₀ and/or an ACE2 Kᵢ not greater than about 1000 nM.

10. The method of claim 1, wherein the ACE2 inhibitor exhibits selectivity for ACE2 versus ACE, as expressed by the ratio of IC₅₀(ACE) to IC₅₀(ACE2), of at least about 10³.

11. The method of claim 1, wherein the ACE2 inhibitor is a compound of the formula 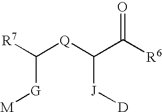
wherein
R⁶ is hydroxyl or a protecting prodrug moiety;
R⁷ is hydrogen, carboxylic acid, ether, alkoxy, an amide, a protecting prodrug moiety, hydroxyl, thiol, heterocyclyl, alkyl or amine;
Q is CH₂, O, NH or NR³, wherein R³ is substituted or unsubstituted C₁₋₅ branched or straight chain alkyl, C₂₋₅ branched or straight chain alkenyl, substituted or unsubstituted acyl, aryl or a C₃₋₈ ring;
G is a covalent bond or a CH₂, ether, thioether, amine or carbonyl linking moiety;
M is heteroaryl, substituted with at least one subanchor moiety comprising a substituted or unsubstituted cycloalkyl or aryl ring, linked thereto through a sublinking moiety (CH₂)ₙ or (CH₂)ₙO(CH₂)ₙ where n is an integer from 0 to 3;
J is a bond or a substituted or unsubstituted alkyl, alkenyl or alkynyl moiety; and
D is alkyl, alkenyl, alkynyl, aryl or heteroaryl, optionally linked to G or M to form a ring;
or a pharmaceutically acceptable salt or prodrug thereof.

12. The method of claim 11, wherein the ACE2 inhibitor comprises a compound in the (S,S)-configuration selected from the group consisting of
2-[1-carboxy-2-[3-(4-trifluoromethylbenzyl)-3H-imidazol-4-yl]ethylamino]-4-methyl-pentanoic acid;
2-[1-carboxy-2-[3-naphthalen-1-ylmethyl-3H-imidazol-4-yl]ethylamino]-4-methyl-pentanoic acid;
2-[1-carboxy-2-[3-(4-chlorobenzyl)-3H-imidazol-4-yl]ethylamino]-4-methylpentanoic acid;
2-[1-carboxy-2-[3-(3,4-dichlorobenzyl)-3H-imidazol-4-yl]ethylamino]-4-methyl-pentanoic acid;
2-[1-carboxy-2-[3-(4-cyanobenzyl)-3H-imidazol-4-yl]ethylamino]-4-methylpentanoic acid;
2-[1-carboxy-2-[3-(3-chlorobenzyl)-3H-imidazol-4-yl]ethylamino]-4-methylpentanoic acid;
2-[1-carboxy-2-[3-(3,5-dichlorobenzyl)-3H-imidazol-4-yl]ethylamino]-4-methyl-pentanoic acid;
2-[1-carboxy-2-[3-(4-methylbenzyl)-3H-imidazol-4-yl]ethylamino]-4-methylpentanoic acid;
2-[1-carboxy-2-[3-(3,4-dimethylbenzyl)-3H-imidazol-4-yl]ethylamino]-4-methyl-pentanoic acid;
2-[1-carboxy-2-[3-(3-methylbenzyl)-3H-imidazol-4-yl]ethylamino]-4-methylpentanoic acid;
2-[1-carboxy-2-[3-(3,5-dimethylbenzyl)-3H-imidazol-4-yl]ethylamino]-4-methyl-pentanoic acid;
2-[1-carboxy-2-[3-(4-trifluoromethoxybenzyl)-3H-imidazol-4-yl]ethylamino]-4-methyl-pentanoic acid;
2-[1-carboxy-2-[3-(4-isopropylbenzyl)-3H-imidazol-4-yl]ethylamino]-4-methyl-pentanoic acid;
2-[1-carboxy-2-[3-(4-tert-butylbenzyl)-3H-imidazol-4-yl]ethylamino]-4-methyl-pentanoic acid;
2-[1-carboxy-2-[3-(4-nitrobenzyl)-3H-imidazol-4-yl]ethylamino]-4-methylpentanoic acid;
2-[1-carboxy-2-[3-(2,3-dimethoxybenzyl)-3H-imidazol-4-yl]ethylamino]-4-methyl-pentanoic acid;
2-[1-carboxy-2-[3-(2,3-difluorobenzyl)-3H-imidazol-4-yl]ethylamino]-4-methyl-pentanoic acid;
2-[1-carboxy-2-[3-(2,3-dichlorobenzyl)-3H-imidazol-4-yl]ethylamino]-4-methyl-pentanoic acid;
2-[1-carboxy-2-[3-(3-trifluoromethylbenzyl)-3H-imidazol-4-yl]ethylamino]-4-methyl-pentanoic acid;
2-[2-(3-benzo[1,3]dioxol-5-ylmethyl-3H-imidazol-4-yl)-1-carboxylamino]-4-methylpentanoic acid;
2-[1-carboxy-2-[3-(2-cyclohexylethyl)-3H-imidazol-4-yl]ethylamino]-4-methyl-pentanoic acid;
2-[1-carboxy-2-[3-phenethyl-3H-imidazol-4-yl]ethylamino]-4-methylpentanoic acid;
2-[1-carboxy-2-[3-(3-iodobenzyl)-3H-imidazol-4-yl]ethylamino]-4-methylpentanoic acid;
2-[1-carboxy-2-[3-(3-fluorobenzyl)-3H-imidazol-4-yl]ethylamino]-4-methylpentanoic acid;
2-[1-carboxy-2-[3-benzyloxymethyl-3H-imidazol-4-yl]ethylamino]-4-methylpentanoic acid;
2-[1-carboxy-2-[3-(4-butylbenzyl)-3H-imidazol-4-yl]ethylamino]-4-methylpentanoic acid;
2-[1-carboxy-2-[3-(2-methylbenzyl)-3H-imidazol-4-yl]ethylamino]-4-methylpentanoic acid;
2-[1-carboxy-2-[2-phenylthiazol-4-yl]ethylamino]-4-methylpentanoic acid;
2-[1-carboxy-2-[1-benzyl)-1H-pyrazol-4-yl]ethylamino]-4-methylpentanoic acid;
2-[1-carboxy-2-[3-(2-methylbiphenyl-3-ylmethyl)-3H-imidazol-4-yl]ethylamino]-4-methylpentanoic acid;
and pharmaceutically acceptable salts thereof.

13. A method for moderating a gastrointestinal adverse effect induced by exposure to at least one cytotoxic agent in a subject, comprising administering to the subject a therapeutically effective amount of a compound selected from the group consisting of (S,S)-2-[1-carboxy-2-[3-(3,5-dichlorobenzyl)-3H-imidazol-4-yl]-ethylamino]-4-methylpentanoic acid (ORE1001), pharmaceutically acceptable salts thereof and prodrugs thereof.

14. The method of claim 13, wherein the at least one cytotoxic agent comprises ionizing radiation and/or at least one cytotoxic chemical agent.

15. The method of claim 13, wherein the gastrointestinal adverse effect occurs in one or more of the mouth, pharynx, esophagus, stomach, duodenum, jejunum, ileum, cecum, colon and rectum.

16. The method of claim 13, wherein the gastrointestinal adverse effect comprises enteritis, colitis, proctitis and/or oral mucositis.

17. The method of claim 13, wherein the subject is a cancer patient, and said exposure results from a course of anticancer therapy with radiation and/or at least one cytotoxic chemotherapeutic agent administered to the patient.

18. The method of claim 17, wherein administration of said compound begins before the first dose of radiation or chemotherapeutic agent in the course of anticancer therapy.

19. The method of claim 18, wherein administration of said compound begins about 1 to about 30 days before said first dose of radiation or chemotherapeutic agent.

20. The method of claim 13, wherein said compound is administered to the subject in an amount of about 0.5 to about 5000 mg/day.

21. The method of claim 13, wherein said compound is ORE1001 or a pharmaceutically acceptable salt thereof.

22. A method for treating a cancerous condition in a subject, comprising administering to the subject (a) at least one cytotoxic anticancer agent and (b) an ACE2 inhibitor in an amount effective to reduce incidence or severity of a gastrointestinal adverse effect induced by the anticancer agent.

23. The method of claim 22, wherein the at least one anticancer agent is administered in a regimen comprising at least one course of therapy, and wherein administration of the ACE2 inhibitor begins before the first dose of anticancer agent in a course of therapy.

24. The method of claim 23, wherein administration of the ACE2 inhibitor begins about 1 to about 30 days before said first dose of anticancer agent.

25. The method of claim 22, wherein the at least one anticancer agent comprises ionizing radiation and/or at least one cytotoxic chemotherapeutic agent.

26. The method of claim 22, wherein the at least one anticancer agent comprises at least one cytotoxic chemotherapeutic agent selected from the group consisting of alkylating agents, platinum complexes, antimetabolites, antimitotic agents, topoisomerase inhibitors, intercalating agents and combinations thereof.

27. The method of claim 22, wherein the ACE2 inhibitor exhibits selectivity for ACE2 versus ACE, as expressed by the ratio of IC₅₀(ACE) to IC₅₀(ACE2), of at least about 10³.

28. A method for treating a cancerous condition in a subject, comprising administering to the subject (a) at least one cytotoxic anticancer agent and (b) a compound selected from the group consisting of ORE1001, pharmaceutically acceptable salts thereof and prodrugs thereof in an amount effective to moderate a gastrointestinal adverse effect induced by the anticancer agent.

29. The method of claim 28, wherein the at least one anticancer agent is administered in a regimen comprising at least one course of therapy, and wherein administration of said compound begins before the first dose of anticancer agent in a course of therapy.

30. The method of claim 29, wherein administration of said compound begins about 1 to about 30 days before said first dose of anticancer agent.

31. The method of claim 28, wherein the at least one anticancer agent comprises ionizing radiation and/or at least one cytotoxic chemotherapeutic agent.

32. The method of claim 28, wherein the at least one anticancer agent comprises at least one cytotoxic chemotherapeutic agent selected from the group consisting of alkylating agents, platinum complexes, antimetabolites, antimitotic agents, topoisomerase inhibitors, intercalating agents and combinations thereof.

33. The method of claim 28, wherein said compound is ORE1001 or a pharmaceutically acceptable salt thereof.

34. A therapeutic combination comprising (a) at least one cytotoxic anticancer agent and (b) an ACE2 inhibitor in an amount effective, when administered to a subject receiving the anticancer agent, to moderate a gastrointestinal adverse effect induced in the subject by the anticancer agent.

35. The combination of claim 34, wherein the at least one anticancer agent is selected from the group consisting of ionizing radiation, alkylating agents, platinum complexes, antimetabolites, antimitotic agents, topoisomerase inhibitors, intercalating agents and combinations thereof.

36. The combination of claim 34, wherein the ACE2 inhibitor exhibits selectivity for ACE2 versus ACE, as expressed by the ratio of IC₅₀(ACE) to IC₅₀(ACE2), of at least about 10³.

37. A therapeutic combination comprising (a) at least one cytotoxic anticancer agent and (b) a compound selected from the group consisting of ORE1001, pharmaceutically acceptable salts thereof and prodrugs thereof in an amount effective, when administered to a subject receiving the anticancer agent, to moderate a gastrointestinal adverse effect induced in the subject by the anticancer agent.

38. The combination of claim 37, wherein the at least one anticancer agent is selected from the group consisting of ionizing radiation, alkylating agents, platinum complexes, antimetabolites, antimitotic agents, topoisomerase inhibitors, intercalating agents and combinations thereof.

39. The combination of claim 37, wherein said compound is ORE1001 or a pharmaceutically acceptable salt thereof.
